SEQUENCE LISTING (21 sequences)

SEQ ID NO: 1 - DNA, 21 bp, synthetic construct
  source 1..21: mol_type = other DNA
tcgcaacgttgccttcgaagg

SEQ ID NO: 2 - DNA, 26 bp, synthetic construct
  source 1..26: mol_type = other DNA
tcgcgtgacgtcatcgatcgatcggg

SEQ ID NO: 3 - DNA, 26 bp, synthetic construct
  source 1..26: mol_type = other DNA
tcgtacgtaggcgcaatcgattgggg

SEQ ID NO: 4 - DNA, 25 bp, synthetic construct
  source 1..25: mol_type = other DNA
tcgatcgcacgtgccgtcgacggcg

SEQ ID NO: 5 - DNA, 25 bp, synthetic construct
  source 1..25: mol_type = other DNA
tcggggacgtcaagtcgacttgggg

SEQ ID NO: 6 - DNA, 24 bp, synthetic construct
  source 1..24: mol_type = other DNA
tcgcgtacgtcggtcgatcgcgcg

SEQ ID NO: 7 - DNA, 24 bp, synthetic construct
  source 1..24: mol_type = other DNA
tcgctacgtgcggatcgagcgccg

SEQ ID NO: 8 - DNA, 23 bp, synthetic construct
  source 1..23: mol_type = other DNA
tcgacgcacgtgggcctcgaggg

SEQ ID NO: 9 - DNA, 23 bp, synthetic construct
  source 1..23: mol_type = other DNA
tcgcgtcggacgtgcgtcgacgg

SEQ ID NO: 10 - DNA, 22 bp, synthetic construct
  source 1..22: mol_type = other DNA
tcgtacgtacgccggtcgaccg

SEQ ID NO: 11 - DNA, 22 bp, synthetic construct
  source 1..22: mol_type = other DNA
tcgctacgtgagtcgactcggg

SEQ ID NO: 12 - DNA, 21 bp, synthetic construct
  source 1..21: mol_type = other DNA
tcgttgacgtcgcatcgatcg

SEQ ID NO: 13 - DNA, 21 bp, synthetic construct
  source 1..21: mol_type = other DNA
tcgcacgtgccgtcgacgccg

SEQ ID NO: 14 - DNA, 20 bp, synthetic construct
  source 1..20: mol_type = other DNA
tcggacgtcgcagtcgacgg

SEQ ID NO: 15 - DNA, 19 bp, synthetic construct
  source 1..19: mol_type = other DNA
tcgtacgtacgtcgacggg

SEQ ID NO: 16 - DNA, 20 bp, synthetic construct
  source 1..20: mol_type = other DNA
gggggacgatcgtcgggggg

SEQ ID NO: 17 - DNA, 24 bp, synthetic construct
  source 1..24: mol_type = other DNA
tcgtcgttttgtcgttttgtcgtt

SEQ ID NO: 18 - DNA, 22 bp, synthetic construct
  source 1..22: mol_type = other DNA
tgactgtgaacgttcgagatga

SEQ ID NO: 19 - DNA, 22 bp, synthetic construct
  source 1..22: mol_type = other DNA
tcgtcgttttcggcgcgcgccg

SEQ ID NO: 20 - DNA, 29 bp, synthetic construct
  source 1..29: mol_type = other DNA
tggccaagcttgggccccttgcaagggcc

SEQ ID NO: 21 - protein, 546 aa, synthetic construct
MGTVNKPVVGVLMGFGIITGTLRITNPVRASVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSTCLYHPNAPQCLSHMNSGCTFTSPHLAQRVASTVYQNCEHADNYTAYCLGISHMEPSFGLILHDGGTTLKFVDTPESLSGLYVFVVYFNGHVEAVAYTVVSTVDHFVNAIEERGFPPTAGQPPATTKPKEITPVNPGTSPLIRYAAWTGGLA

(19) United States
(12) Patent Application Publication
(10) Pub. No.: US 2026/0232792 A1
(43) Pub. Date: Aug. 13, 2026

(54) CPG OLIGONUCLEOTIDE AND USE THEREOF

(71) Applicant: PARR BIOTECHNOLOGY (HEBEI) CO., LTD., Shijiazhuang (CN)

(72) Inventor: Ligong WANG, Shijiazhuang (CN)

(21) Appl. No.: 19/149,138

(22) PCT Filed: Jan. 17, 2024
(86) PCT No.: PCT/CN2024/072835

§ 371 Date: Jul. 18, 2025

(30) Foreign Application Priority Data
Jan. 19, 2023 (CN) 202310066663.2

(51) Int. Cl. A61K 39/25; A61K 39/00; A61K 39/02; A61K 39/145; A61K 39/215; A61K 39/29; A61K 39/39; A61K 39/395; A61P 31/22; C12N 15/113

(52) U.S. Cl. CPC A61K 39/25; A61K 39/0002; A61K 39/0011; A61K 39/02; A61K 39/145; A61K 39/215; A61K 39/29; A61K 39/39; A61K 39/39558; A61P 31/22; C12N 15/113; A61K 2039/53; A61K 2039/54; A61K 2039/55505; C12N 2310/3125; C12N 2310/315; C12N 2310/3521

(57) ABSTRACT

A CpG oligonucleotide and use of the CpG oligonucleotide are described. The CpG oligonucleotide has TLR9 agonist activity.

### CROSS-REFERENCE TO RELATED APPLICATIONS

This application claims priority to Patent Application No. CN202310066663.2, filed on Jan. 19, 2023, and entitled “CPG OLIGONUCLEOTIDE AND USE THEREOF”, which is incorporated herein by reference in its entirety.

### TECHNICAL FIELD

The present invention generally relates to the field of biomedical technologies, and specifically, to a CpG oligonucleotide and use thereof.

### BACKGROUND

Toll-like receptors (TLR) are transmembrane proteins that mainly act as biosensors of microbial components to identify bacterial and viral components. TLRs constitute a main defense mechanism in mammalian infections and some non-infectious diseases. 10 human TLRs (TLR 1 to TLR 10) and 12 mouse TLRs (TLR 1 to TLR9 and TLR 11 to TLR 13) have been identified by now. TLR9 is expressed in human plasmacytoid dendritic cells (pDC) and B cells, as well as various mouse myeloid lineage cells (including monocytes, macrophages, and conventional dendritic cells).

TLR9 specifically recognizes CpG motifs included in bacteria, viruses, plasmids, or synthetic double-stranded or single-stranded oligonucleotides. CpG ODN is an immunomodulatory synthetic oligonucleotide specifically designed for stimulating TLR9. Four types of synthetic CpG ODNs, i.e., type-K (also known as type-B) ODN, type-D (also known as type-A) ODN, type-C ODN, and type-P ODN, have been depicted by now, and each of these types has a different structure and biological attribute.

Currently, the only approved CpG ODN is CpG 1018, which is contained as adjuvant in a few types of vaccines, and only vaccines for hepatitis B and novel coronavirus have been approved. In addition, no CpG ODNs have been approved for treating cancers by now. In view of heavy burdens of infectious diseases and cancers across the globe, there is still a need to develop TLR9 agonists with better activity to be applied in prevention and treatment of infectious diseases and cancers.

### SUMMARY

An aspect of the present invention relates to a CpG oligonucleotide having a sequence as set forth in SEQ ID NO: 1.

Another aspect of the present invention relates to an immunostimulatory composition comprising the above CpG oligonucleotide.

A still another aspect of the present invention relates to a delivery system, comprising: (i) the above CpG oligonucleotide or the above immunostimulatory composition; and (ii) a delivery medium.

A yet another aspect of the present invention relates to use of the above CpG oligonucleotide, the above immunostimulatory composition, or the above delivery system in preparation of a medicant for regulating immune cell activity, where the use is conducted in vivo or in vitro.

A still yet another aspect of the present invention relates to use of the above CpG oligonucleotide, the above immunostimulatory composition, or the above delivery system in preparation of a medicant for treating and/or preventing at least one indication, selected from a tumor, a viral infection, a bacterial infection, a fungal infection, a parasitic infection, a chemotherapy side effect, fatigue, or immunosuppression, as well as a low immune response of the subject to an antigen, in a subject in need thereof.

A further aspect of the present invention relates to a method for inducing a TLR9-mediated immune response in a subject, comprising administering an effective amount of the above CpG oligonucleotide, the above immunostimulatory composition, or the above delivery system to the subject.

The novel CpG oligonucleotide provided in the present invention has TLR9 agonist activity and has better medicinal activity than its counterparts in the prior art. The CpG oligonucleotide can broadly regulate immune responses, increase the release of cytokines such as IFN-α, IL-6, and TNF-α, and have antitumor and anti-infection capabilities, and therefore, can be applied in fields such as antitumor drugs and vaccines.

### BRIEF DESCRIPTION OF DRAWINGS

To describe the technical solutions in the specific embodiments of the present invention or in the prior art more clearly, the following briefly describes the accompanying drawings required for describing the specific embodiments or the prior art. Apparently, the accompanying drawings in the following description show merely some embodiments of the present invention, and persons of ordinary skills in the art may still derive other drawings from these accompanying drawings without inventive efforts.

FIG. 1 indicates that CpG1 provided in an embodiment of the present invention has an effect of activating mouse TLR9. HEK-Blue mTLR9 cells (180 μL, 2×10⁵ to 3×10⁵ cells/mL) were inoculated in a 96-well plate and cultured for about 1-24 hours. Then 20 μL of different sequence solutions (CpG1, CpG 2395, CpG 7909, and CpG 1018, and FX-700 as a negative control) were added with final concentrations of 0, 0.25, 0.5, 1, 2, 5, 10, 20, and 40 μM, and the cells were cultured for 24 hours. The culture supernatant was collected, QUANTI-Blue™ solution was added, and the cells were further incubated for 15 minutes. The OD value at 630 nm was measured by using a spectrophotometer to quantify the level of secreted embryonic alkaline phosphatase (SEAP). The SEAP secretion levels of the HEK-Blue™ mTLR9 cell line were measured to evaluate activation effects of different sequences at different concentrations on mouse TLR9.

FIG. 2 indicates that CpG1 provided in an embodiment of the present invention has the capability to induce proliferation of mouse spleen cells. Mouse spleen cells (2×10⁶ cells/100 μL) were inoculated in a 96-well plate and cultured for about 1-24 hours. Then 10 μL of different sequence solutions (CpG1, CpG 2395, CpG 7909, and CpG 1018, and FX-700 as a negative control) were added with final concentrations of 0, 0.0156, 0.03125, 0.0625, 0.125, 0.25, 0.5, 1, and 2 μM, and the cells were cultured for about 72 hours. 10 μL of CCK-8 solution was added to each well and the cells were further incubated for 1-4 hours. The OD value at 450 nm was measured via a microplate reader, which indirectly reflected the number of viable cells.

FIG. 3 indicates that sequence 1 effectively stimulates human PBMCs to secret IFN-α. Human PBMCs (1×10⁶ to 2×10⁶ cells/mL) were inoculated in a 96-well plate and cultured for at least 1 hour. Then 10 μL of different sequence solutions (CpG1, CpG 2395, CpG 7909, and CpG 1018, and FX-700 as a negative control) were added with final concentrations of 0, 0.0156, 0.03125, 0.0625, 0.125, 0.25, 0.5, 1, 2, 5, and 10 μM, and the cells were cultured for 16 to 24 hours. Cell supernatant was taken and a content of IFN-α was measured via a kit.

FIG. 4 indicates that CpG1 effectively stimulates human PBMCs to secret IL-6. Human PBMCs (1×10⁶ to 2×10⁶ cells/mL) were inoculated in a 96-well plate and cultured for at least 1 hour. Then 10 μL of different sequence solutions (CpG1, CpG 2395, CpG 7909, and CpG 1018, and FX-700 as a negative control) were added with final concentrations of 0, 0.0156, 0.03125, 0.0625, 0.125, 0.25, 0.5, 1, 2, 5, and 10 μM, and the cells were cultured for 16 to 24 hours. Cell supernatant was taken and a content of IL-6 was measured via a kit.

FIG. 5 indicates that CpG1 effectively stimulates human PBMCs to secret TNF-α. Human PBMCs (1×10⁶ to 2×10⁶ cells/mL) were inoculated in a 96-well plate and cultured for at least 1 hour. Then 10 μL of different sequence solutions (CpG1, CpG 2395, CpG 7909, and CpG 1018, and FX-700 as a negative control) were added with final concentrations of 0, 0.0156, 0.03125, 0.0625, 0.125, 0.25, 0.5, 1, 2, 5, and 10 μM, and the cells were cultured for 16 to 24 hours. Cell supernatant was taken and a content of TNF-α was measured via a kit.

FIG. 6 indicates that CpG1 effectively enhances immunogenicity of a novel coronavirus vaccine composition. Six- to eight-week-old female BALB/c mice were used, each group of mice were immunized once by intramuscular injection on D0 and D28, separately, each injection volume was 200 μL, and the mice were given injections of saline, a combination of BA 4/5 and aluminum hydroxide, and a combination of BA 4/5, aluminum hydroxide, and CpG 1 for immunization separately. Mouse blood was sampled at specified time points, serum was obtained by centrifugation, and antibody titers of the blood samples were measured by indirect ELISA.

FIG. 7 indicates that CpG1 effectively enhances immunogenicity of a varicella-zoster virus vaccine composition. Female C57BL/6J mice were used, each group of mice were immunized once by intramuscular injection on D0 and D28, separately, each injection volume was 50 μL, and the mice were given injections of PBS solution, 5 μg of gE, a combination of 5 μg of gE and different amounts of CpG 1, and a combination of 5 μg of gE, 8 μg of CpG 1, and 40 μg of Alum for immunization separately. Mouse blood was sampled at specified time points, serum was obtained by centrifugation, and antibody titers of the blood samples were measured by ELISA.

FIG. 8 indicates that CpG1 effectively enhances immunogenicity of a hepatitis B vaccine composition. Female BALB/c mice were used, each group of mice were immunized once by intraperitoneal injection on D0 and D28, separately, each injection volume was 1 mL, and the mice were given injections of PBS solution, hepatitis B antigen, and a combination of hepatitis B antigen, aluminum hydroxide, and different amounts of CpG 1 for immunization separately. Mouse blood was sampled at specified time points, serum was obtained by centrifugation, and antibody levels of the blood samples were measured by the kit.

FIGS. 9A to 9D indicate that CpG1 effectively enhances immunogenicity of quadrivalent influenza split vaccines, namely, types A1, A3, By, and Bv influenza vaccine compositions. Female BALB/c mice were used, each group of mice were immunized once by injection at the inner thighs on DO, the injection volume was 0.1 mL, and the mice were given injections of PBS solution, influenza antigen, and a combination of influenza antigen and different amounts of CpG 1 for immunization separately. Mouse blood was sampled at specified time points, serum was obtained by centrifugation, and antibody levels of the blood samples were measured by ELISA.

### DETAILED DESCRIPTION

Reference is made in detail to embodiments of the present invention, and one or more examples are described below. Each example is provided to illustrate other than limit the present invention. Actually, apparently, persons skilled in the art can make various modifications and variations to the present invention without departing from the scope or spirit of the present invention. For example, features illustrated or described as a part of an embodiment can be used in another embodiment to create still another embodiment.

Unless otherwise specified, all terms (including technical and scientific terms) used to disclose the present invention have the same meanings as commonly understood by persons of ordinary skills in the art to which the present invention belongs. With reference to further guidance, the following definitions are used to better understand teachings of the present invention. The terms used herein in the specification of the present invention are only for a purpose of describing specific examples, and are not intended to limit the present invention.

### Description of Terms

A selection range of a term “and/or” used herein includes any one of two or more related listed items, and any combinations or all combinations of the related listed items, and the said combinations include combinations of any two or more related listed items or all the related listed items. It should be noted that when at least three items are conjoined by “and/or”, it should be understood that, in this application, technical solutions undoubtedly include technical solutions logically conjoined by “and”, and also undoubtedly include technical solutions logically conjoined by “or”. For example, “A and/or B” includes three parallel solutions: A, B, and both A and B. For another example, technical solutions of “A, B, C and/or D” include any one of A, B, C or D (namely, a technical solution logically conjoined by “or”), and also include any combinations and all combinations of A, B, C and D, that is, a combination of any two or three of A, B, C and D and a combination of four of A, B, C and D (namely, technical solutions logically conjoined by “and”).

Terms “comprise”, “include”, and “contain” used in the present invention are synonymous, and are inclusive or open-ended, and do not exclude additional uncited members, elements or method steps.

A value range in present invention that is defined by endpoints includes all values, fractions and the cited endpoints subsumed within the range.

A concentration value involved in the present invention may fluctuate within a specific range. For example, the concentration value may fluctuate within a corresponding precision range. For example, the precision range of 2% allows fluctuation within a range of ±0.1%. A larger fluctuation is also allowed for a larger value or a value that does not require precise control. For example, a precision range of 100 mM can allow fluctuation within a range of ±1%, ±2%, ±5%, and so on. Molecular weight is allowed fluctuation of ±10%.

In the present invention, descriptions such as “multiple” and “various” refer to two or more than two unless otherwise specified.

In the present invention, technical features in open-ended descriptions include closed-ended technical solutions composed of listed features, and also include open-ended technical solutions including the listed features.

As used herein, the terms “nucleic acid”, “nucleic acid molecule”, “nucleotide sequence” and “polynucleotide” refer to linear or branched, single-stranded or double-stranded RNA or DNA, or a hybrid thereof. The term also includes a hybrid of RNA and DNA. Heterocyclic bases or nucleic acid bases incorporated into CpG oligonucleotides can be naturally occurring major purine and pyrimidine bases (namely, uracil, thymine, cytosine, adenine, and guanine), and naturally occurring and synthetic modifications of the major bases. Therefore, CpG-C oligonucleotides can include one or more of inosine, 2′-deoxyuridine, and 2-amino-2′-deoxyadenosine. When CpG is produced through synthesis, less common bases can also be used for synthesis. The terms “nucleotide sequence”, “nucleic acid”, “nucleic acid molecule”, “nucleic acid construct”, “oligonucleotide”, “ODN”, and “polynucleotide” may be used interchangeably herein. The nucleic acid molecules and/or nucleotide sequences provided herein are displayed from left to right and from 5′ to 3′ herein, and are denoted as standard codes for representing nucleotide symbols specified in the World Intellectual Property Organization (WIPO) ST.26 standard.

As used herein, the term “TLR9 agonist” generally refers to an oligonucleotide-based compound that can enhance, induce, or regulate TLR9-mediated immune stimulation.

As used herein, the term “CpG or CpG motif” refers to a nucleic acid with cytosine linked by phosphate bonds to guanine at the back, where the pyrimidine ring of cytosine is unmethylated. “Methylated CpG” indicates that the pyrimidine ring of cytosine is methylated, usually at site 5 of the pyrimidine ring. The CpG motif is a base pattern that includes an unmethylated central CpG and at least one base on the 3′ and 5′ sides of the central CpG. Flanking bases of CpG contribute to most activity of CpG ODN.

As used herein, the term “CpG ODN” refers to CpG oligodeoxynucleotide that has about at least ten nucleotides in length and that includes one unmethylated CpG. The CpG ODN is single-stranded. The CpG ODN can be completely or partially unmethylated. CpG ODNs include type D (also known as type A), type K (also known as type B), type C, and type P ODNs.

As used herein, the term “patient”, “subject”, or “body” is used to refer to any animal, in particular, a mammal, and the method in this disclosure can be used to treat any type of poultry, mammalian or aquatic species, including, in particular, human and mammalian veterinary patients such as cattle, sheep, a goat, a horse, a dog, a pig, a cat, a panda, an elephant, a rabbit, a rat, or a mouse.

As used herein, “antigen” (Ag) refers to any substance that can induce an immune response in the body, that is, a substance that can be specifically recognized by and bound to the antigen receptor (TCR/BCR) on the surface of T/B lymphocyte to activate T/B cells to proliferate and differentiate, thereby producing immune response products (primed lymphocytes or antibodies), and that can specifically bind to the corresponding products in vivo and in vitro, including, but not limited to, an xenoantigen, for example a pathogenic microorganism, a virus, a toxoid, and other antigens of different species; and an allotypic antigen, an autoantigen, and a heterophilic antigen.

As described herein, the term “antibodies” include a polyclonal antibody and a monoclonal antibody, and the term “antibody fragment” includes antigen-binding fragments of these antibodies, including Fab, F(ab′)₂, Fd, Fv, Fab′-SH, scFv, bispecific antibodies, and minimum recognition units of the antibodies, and single-chained derivatives of these antibodies and fragments, such as scFv-Fc. The antibodies can be selected from IgG1, IgG2, IgG3, IgG4, IgA, IgM, IgE, and IgD. In addition, the term “antibodies” include naturally occurring antibodies and non-naturally occurring antibodies, including, for example, chimeric, bifunctional, humanized and human antibodies, and related synthesized isoforms of the antibodies. The term “antibody” is interchangeable with “immunoglobulin”.

All documents mentioned in the present invention are cited as references in this application, as if each document were individually cited as reference. Unless in conflict with the inventive objective and/or the technical solution of this application, the cited references involved in the present invention are cited in their entireties and for all the objectives. When the present invention involves references, definitions of a relevant technical characteristic, term, noun, phrase, and the like in the references are also cited. When the present invention involves references, examples and preferred embodiments of the cited relevant technical characteristic may also be incorporated into this application as a reference, provided that the present invention can be practiced. It should be understood that when the cited content conflicts with the description in this application, this application shall prevail or be amended adaptively based on the description of this application.

### DETAILED DESCRIPTION OF INVENTION

### CPG Oligonucleotide

In the present invention, a CpG oligonucleotide with excellent performance was accidentally discovered and then studied.

The first aspect of the present invention relates to a CpG oligonucleotide having a sequence as set forth in SEQ ID NO: 1.

The CpG oligonucleotides herein include one or more CpG oligonucleotides with chemical modifications. Modifications include, but are not limited to, modifications of 3′OH or 5′OH groups, modifications of nucleotide bases, modifications of sugar components, modifications of backbones, and modifications of phosphate groups. With such modifications, CpG oligonucleotides can be more stable and/or less susceptible to degradation under certain conditions. For example, in some embodiments, the CpG oligonucleotides are nuclease-resistant. Such modifications can occur during or after the synthesis of oligonucleotides, and can occur on phosphodiester bridge bonds between nucleosides, on ribose units, and/or on naturally occurring nucleobases (that is, adenine, guanine, cytosine, and thymine). When modifications are made during the synthesis of oligonucleotides, the modified bases can be incorporated into the oligonucleotides or at the ends of the oligonucleotides. When modifications are made after the synthesis of oligonucleotides, such modifications can be carried out via reactive groups, for example, amino-modified components, 3′ or 5′ OH, or phosphate ester groups.

The CpG oligonucleotide may contain naturally occurring or modified non-naturally occurring bases, and may include modified sugars, phosphate esters, and/or terminals. For example, in addition to the phosphodiester linkages, phosphate ester modifications include, but are not limited to, methyl phosphonate, phosphorothioate, phosphoramidate (bridging or non-bridging), phosphotriester, and phosphorodithioate, which can be combined randomly for use. In some embodiments, the CpG oligonucleotide includes only phosphorothioate linkages, only phosphodiester linkages, or a combination of phosphodiester and phosphorothioate linkages.

The CpG oligonucleotide can include one or more ribonucleotides (with ribose as the sole or main sugar component), deoxyribonucleotides (with deoxyribose as the main sugar component), modified sugars or sugar analogs. Therefore, in addition to ribose and deoxyribose, the sugar component can also be pentose, deoxypentose, hexose, deoxyhexose, glucose, arabinose, xylose, lyxose, and a sugar analog such as a cyclopentyl group. The sugar can exist in a pyranosyl or furanosyl form. In the CpG oligonucleotide, the sugar component is preferably a furanoside of ribose, deoxyribose, arabinose, or 2′-O-alkylribose, and the sugar can be ligated to various heterocyclic bases in terminal isomeric configurations.

Known sugar modifications in the field may also be made, for example 2′-alkoxy-RNA analogs, 2′-amino-RNA analogs, 2′-fluoro-DNA, and 2′-alkoxy- or amino-RNA/DNA chimeras, as well as others described herein, to be combined with any phosphate ester modification. Examples of base modifications include, but are not limited to, the addition of electron-withdrawing groups to C-5 and/or C-6 of cytosine (for example, 5-bromocytidine, 5-chlorocytidine, 5-fluorocytidine, or 5-iodocytidine) in the CpG oligonucleotides and to the C-5 and/or C-6 of uridine (for example, 5-bromouridine, 5-chlorouridine, 5-fluorouridine, or 5-iodouridine) in the CpG oligonucleotides. The use of the base modification in a palindromic sequence of the CpG oligonucleotide should not interfere with the self-complementarity of bases involved in Watson-Crick base pairing. However, in addition to the palindromic sequence, modified bases can be used without such restriction. For example, 2′-O-methyl-uridine and 2′-O-methyl-cytidine can be used outside the palindromic sequence. However, 5-bromo-2′-deoxycytidine can be used both inside and outside the palindromic sequence. Other modified nucleotides that can be used both inside and outside the palindromic sequence include 7-deaza-8-aza-dG, 2-amino-dA, and 2-thio-dT.

In some embodiments, the CpG oligonucleotide includes a modification of one or more phosphate ester groups. The modification of phosphate ester group may be understood as substitution of at least part of the nucleotide phosphodiester linkages with phosphorothioate ester linkages in polynucleotide derivatives.

In some embodiments, more than 50%, 60%, 70%, 80%, or 90% of nucleotides in the CpG oligonucleotides as set forth in SEQ ID NO: 1 have modifications of phosphate ester groups.

In some embodiments, the modification of the phosphate ester group includes one or more of an internucleotide phosphorothioate ester linkage, a methylphosphonate linkage, and a boranophosphate linkage.

In some embodiments, the CpG oligonucleotide in this disclosure has a homogeneous backbone (for example, fully phosphodiester or fully phosphorothioate ester) or a heterogeneous (or chimeric) backbone. The modification of phosphorothioate backbone can reduce sensitivity of the oligonucleotide to the nuclease, which makes the oligonucleotide more stable under some conditions (compared with natural nucleic acid with the phosphodiester backbone). Other linkages that can endow the nucleic acids in this disclosure with greater stability include but are not limited to phosphorodithioate linkages, methyl phosphonate linkages, methyl phosphorothioate linkages, boranophosphate linkages, peptide bonds, alkyl bonds, and dephosphorylated bonds. Therefore, in some embodiments, the CpG oligonucleotide has a non-naturally occurring backbone. In some embodiments, the CpG oligonucleotide has a backbone completely made of phosphorothioate.

### Immunostimulatory Composition

A second aspect of the present invention relates to an immunostimulatory composition including the above CpG oligonucleotide.

In some embodiments, the immunostimulatory composition further includes another adjuvant in addition to the CpG oligonucleotide.

In some embodiments, the adjuvant includes one or more of alum, complete Freund's adjuvant, incomplete Freund's adjuvant, squalene, squalane, muramyl dipeptide, MF59, AS03, AS04, 3-O-desacyl-4′-monophosphoryl lipid A, flagellin, Poly(I:C), and aluminum and calcium salts.

Complete Freund's adjuvant, incomplete Freund's adjuvant, squalene, squalane, and alum are usually not applied to humans.

Preferably, the adjuvant includes an aluminum salt or a calcium salt, where the aluminum salt may be aluminum sulfate, aluminum hydroxide, aluminum phosphate, potassium aluminum sulfate, and the like.

In some embodiments, a typical representative product form of this immunostimulatory composition is a vaccine adjuvant.

In some embodiments, the immunostimulatory composition includes 1-2000 μg/mL of the above CpG oligonucleotide; or preferably includes 5-500 μg/mL of the above CpG oligonucleotide. The immunostimulatory composition further includes an aluminum salt, for example an aluminum hydroxide adjuvant. A content of the aluminum salt adjuvant in the immunostimulatory composition is 100 μg/mL to 1 mg/mL, or preferably, 400 μg/mL to 800 μg/mL.

In some embodiments, the immunostimulatory composition also includes at least one antigen.

A typical representative product form of this immunostimulatory composition is a vaccine.

In some embodiments, the vaccine is a water-in-oil emulsion with aqueous and oil phases.

In some embodiments, the vaccine is an oil-in-water emulsion with aqueous and oil phases.

The vaccine is typically prepared for parenteral administration. Typical immunizations are implemented via a nasal route. However, oral and subcutaneous (SC) routes, and an intramuscular (IM), intravenous (IV), intraperitoneal (IP), or intradermal (ID) injection are also considered in the present invention.

The vaccine is administered appropriate to the dosage and formulation, for example, therapeutically and immunogenically effective amount. The amount depends on an object under treatment, an antibody synthesization capability of the immune system of the subject, and an expected degree of protection. An exact amount of active component to be administered is at discretion of a physician and the amount varies from individual to individual. Appropriate treatments for initial administration and booster vaccination can also be changed, but the first administration is typically followed by 1 injection or administration in another method after a certain period of time (several weeks or months).

In some embodiments, the antigen is a tumor antigen, a viral antigen, a bacterial antigen, a fungal antigen, or a parasitic antigen.

Herein, the exemplary tumor antigens are well-known to persons skilled in the art, including tumor-specific antigens (TSA) and tumor-associated antigens (TAA), for example, any one or more selected from the group consisting of the following antigens or functional fragments thereof: α-fetoprotein (AFP), α-actinin-4, A3, antigen specific to A33 antibodies, ART-4, B7, Ba 733, BAGE, BrE3 antigen, BMCA, CA125, CAMEL, CAP-1, carbonic anhydrase IX, CASP-8/m, CCL19, CCL21, CD1, CD1a, CD2, CD3, CD4, CD5, CD8, CD11A, CD14, CD15, CD16, CD18, CD19, CD21, CD23, CD25, CD29, CD30, CD32b, CD37, CD44, CD45, CD46, CD52, CD54, CD55, CD59, CD64, CD66a-e, CD67, CD70, CD70L, CD74, CD79a, CD79b, CD80, CD83, CD95, CD126, CD132, CD133, CD138, CD147, CD154, CDC27, CDK-4/m, CDKN2A, CLDN family proteins, CXCR4, CXCR7, CXCL12, HIF-1α, colon-specific antigen p (CSAp), CEA (CEACAM-5), CEACAM-6, c-Met, DAM, EGFR, EGFRVIII, EGP-1 (TROP-2), EGP-2, ELF2-M, Ep-CAM, fibroblast activation protein α (FAP), fibroblast growth factor (FGF), Flt-1, Flt-3, folate receptor, G250 antigen, GAGE, GD2, gp100, GRO-B, HLA-DR, HM1.24, human chorionic gonadotropin (HCG) and a subunit thereof, HMGB-1, hypoxia-inducible factor (HIF-1), HSP70-2M, HST-2, Ia, IGF-1R, IFN-γ, IFN-α, IFN-β, IFN-λ, IL-4R, IL-6R, IL-13R, IL-15R, IL-17R, IL-18R, IL-2, IL-6, IL-8, IL-12, IL-15, IL-17, IL-18, IL-23, IL-25, insulin-like growth factor 1 (IGF-1), KC4 antigen, KS-1 antigen, KS1-4, Le-Y, LDR/FUT, macrophage migration inhibitory factor (MIF), MAGE, MAGE-3, MART1, MART-2, NY-ESO-1, TRAG-3, mCRP, MCP-1, MIP-1a, MIP-1β, MIF, MUC1, MUC2, MUC3, MUC4, MUC5ac, MUC13, MUC16, MUM-1/2, MUM-3, NCA66, NCA95, NCA90, mesothelin (MSLN), mucin in pancreatic cancer, prostate stem cell antigen (PSCA), placenta growth factor, p53, PLAGL2, prostatic acid phosphatase, trophoblast cell surface antigen 2 (TROP2), PSA, PRAME, PSMA, PIGF, ILGF, ILGF-1R, IL-6, IL-25, RS5, RANTES, T101, SAGE, S100, survivin, survivin-2B, TAC, TAG-72, tenascin, TRAIL receptor, TNF-α, Tn antigen, Thomsen-Friedenreich antigen, tumor necrosis antigen, VEGFR, ED-B fibronectin, WT-1, 17-1A antigen, complement factor C3, C3a, C3b, C5a, C5, angiogenesis markers, bc1-2, bc1-6, Kras, a carcinogenic gene marker, and a carcinogenic gene product. More preferred solid tumor-specific antigens are selected from the CLDN family proteins, Ep-CAM, FAP, PSCA, TROP2, and MSLN. The CLDN family proteins can be selected from CLDN1, CLDN2, CLDN3, CLDN4, CLDN5, CLDN6, CLDN7, CLDN8, CLDN9, CLDN10, CLDN11, CLDN12, CLDN15, CLDN16, CLDN18 (CLDN18.1 or CLDN18.2), CLDN20, and CLDN23.

In some embodiments, the tumor is cancer, sarcoma, myeloma, leukemia, lymphoma, and a mixed tumor. Non-restrictive examples of tumors that can be treated by the method and composition described herein include cancer cells originated from the following: a bladder, blood, a bone, bone marrow, a brain, an esophagus, a gastrointestinal tract, a gingiva, a head, a kidney, a liver, a lung, a nasopharynx, a neck, an ovary, a prostate, skin, a stomach, a testis, a tongue, or a uterus. In addition, the cancer may specifically belong to, but is not limited to, the following histological types: malignant neoplasm; carcinoma; undifferentiated carcinoma; giant cell carcinoma and spindle cell carcinoma; small cell carcinoma; papillary carcinoma; squamous cell carcinoma; lymphoepithelioma; basal cell carcinoma; pilomatrix carcinoma; transitional cell carcinoma; papillary transitional cell carcinoma; adenocarcinoma; malignant gastrinoma; cholangiocarcinoma; mixed hepatocellular carcinoma and cholangiocarcinoma; trabecular adenocarcinoma; adenoid cystic carcinoma; adenocarcinoma in adenomatous polyps; familial adenomatous polyposis-associated adenocarcinoma; malignant carcinoid tumors; bronchioloalveolar adenocarcinoma; papillary adenocarcinoma; chromophobe carcinoma; eosinophilic carcinoma; eosinophilic adenocarcinoma; basophilic carcinoma; clear cell adenocarcinoma; granular cell tumor; follicular adenocarcinoma; papillary and follicular adenocarcinoma; non-encapsulated sclerosing carcinoma; adrenal cortical carcinoma; skin appendage carcinoma; apocrine carcinoma; sebaceous carcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; invasive ductal carcinoma; medullary carcinoma; lobular carcinoma; inflammatory carcinoma; Paget's disease of the breast; acinar cell carcinoma; adenosquamous carcinoma; adenocarcinoma with squamous differentiation; malignant thymoma; malignant ovarian stromal tumor; malignant thecoma; malignant granular cell tumor; and malignant fibroblastoma; Sertoli cell tumor; malignant testicular interstitial cell tumor; malignant lipid cell tumor; malignant paraganglioma; malignant extramammary paraganglioma; pheochromocytoma; glomangiosarcoma; amelanotic melanoma; superficial spreading melanoma; malignant melanoma in giant pigmented nevus; epithelioid cell melanoma; malignant blue nevus; sarcoma; fibrosarcoma; malignant fibrous histiocytoma; myxosarcoma; liposarcoma; leiomyosarcoma; rhabdomyosarcoma; embryonal rhabdomyosarcoma; alveolar rhabdomyosarcoma; stromal sarcoma; malignant mixed tumor; Müllerian duct mixed tumor; Wilms tumor; hepatoblastoma; carcinosarcoma; malignant mesenchymoma; malignant Brenner tumor; malignant phyllodes tumor; synovial sarcoma; malignant mesothelioma; dysgerminoma; embryonal carcinoma; malignant teratoma; malignant struma ovarii; choriocarcinoma; malignant mesonephroma; angiosarcoma; malignant haemangioendothelioma; Kaposi's sarcoma; malignant hemangiopericytoma; lymphangiosarcoma; osteosarcoma; parosteal osteosarcoma; chondrosarcoma; malignant chondroblastoma; mesenchymal chondrosarcoma; giant cell tumor of bone; Ewing sarcoma; malignant odontogenic tumor; ameloblastic odontosarcoma; malignant ameloblastoma; ameloblastic fibrosarcoma; malignant pinealoma; chordoma; malignant glioma; Ependymoma; astrocytoma; protoplasmic astrocytoma; fibrous astrocytoma; astroblastoma; primitive neuroectodermal al tumor; cerebellar sarcoma; ganglion neuroblastoma; neuroblastoma; retinoblastoma; olfactory neurogenic tumor; malignant meningioma; neurofibrosarcoma; malignant schwannomas; malignant granular cell tumor; paragranuloma; small lymphocytic malignant lymphoma; granuloma fungoides; other specified non-Hodgkin lymphoma; malignant histiocytosis; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryocytic leukemia; myeloid sarcoma; plasmacytoma, rectal cancer, and hairy cell leukemia.

Herein, the exemplary virus may include one or more of adenoviridae, arenaviridae, astroviridae, bunyaviridae, caliciviridae, flaviviridae, hepatitis delta virus, hepeviridae, mononegavirales, nidovirales, picornaviridae, orthomyxoviridae, papillomaviridae, parvoviridae, polyomaviridae, poxviridae, reoviridae, retroviridae, coronaviridae, paramyxovirinae, herpesviridae, or togaviridae.

The exemplary virus also includes a double-stranded DNA virus (dsDNA) such as adenovirus, herpes virus, or poxvirus; single-stranded DNA virus (ssDNA) such as parvovirus; a double-stranded RNA virus (dsRNA) such as rotavirus or reovirus; a positive-sense single-stranded RNA virus (+ssRNA) such as novel coronavirus, norovirus, hepatitis C, hepatitis A virus, or dengue fever, etc.; an antisense single-stranded RNA virus (-ssRNA) such as influenza virus or rabies virus, etc.; a single-stranded RNA retrovirus (ssRNA-RT) such as HIV virus; or a double-stranded DNA retrovirus (dsDNA-RT) such as hepatitis B virus, etc.

When the immunostimulatory composition is a vaccine, the viral antigen can be coronavirus antigen, norovirus antigen, Ebola virus antigen, HIV antigen, influenza virus antigen, rabies virus antigen, herpes virus antigen, rotavirus antigen, hepatitis virus antigen, HIV antigen, HPV antigen, or RSV antigen. Specifically, the viral antigen can be novel coronavirus antigen, varicella-zoster virus antigen, hepatitis B virus antigen, influenza A virus antigen, and influenza B virus antigen. The influenza virus antigen can be type A1 influenza virus antigen, type A3 influenza virus antigen, type Bv influenza virus antigen, or type By influenza virus antigen.

In an embodiment, a mass ratio of the viral antigen to the CpG oligonucleotide in the vaccine is 10:1 to 1:150, or preferably, 5:1 to 1:50.

In an embodiment, the vaccine includes 1-2000 μg/mL of the CpG oligonucleotide and 1-100 μg/mL of the viral antigen; or preferably, includes 5-500 μg/mL of the above CpG oligonucleotide.

Herein, the exemplary bacterium may include one or more of Staphylococcus, Streptococcus, Listeria sp., Erysipelothrix sp., Renibacterium sp., Bacillus sp., Clostridium sp., Mycobacterium sp., Actinobacterium sp., Nocardia sp., Corynebacterium sp., or Rhodococcus sp.; and may further include one or more of Bacillus anthracis, Erysipelothrix, Tetanus bacillus, Listeria bacillus, Clostridium chauvoei, Mycobacterium tuberculosis, Escherichia coli, Bacillus proteus, Dysentery bacillus, Pneumobacillus, Brucella, Clostridium perfringens, Haemophilus influenzae, Haemophilus parainfluenzae, Moraxella catarrhalis, Acinetobacter sp., Yersinia sp., Legionella pneumophila, Bacillus pertussis, Bacillus parapertussis, Shigella sp., Pasteurella sp., Vibrio cholerae, and Vibrio parahaemolyticus.

Herein, the exemplary fungus may include one or more of Coccidioides immitis, Coccidioides posadasii, Histoplasma capsulatum, Histoplasma duboisii, Loboa loboi, Paracoccidioides brasiliensis, Blastomyces dermatitidis, Sporothrix schenckii, Penicillium marneffei, Candida albicans, Candida glabrata, Candida tropicalis, Candida lusitaniae, Aspergillus, Exophiala jeanselmei, Fonsecaea pedrosoi, Fonsecaea compacta, Phialophora verrucosa, Exophiala dermatitidis, Geotrichum candidum, Pseudallescheria boydii, Cryptococcus neoformans, Trichosporon, Rhizopus oryzae, Mucor indica, Absidia corymbifera, Syncephalastrum racemosum, Basidiobolus ranarum, Conidiobolus coronatus, Conidiobolus incongruus, Rhinosporidium seeberi, Hyalohyphomycosis fungus, and Phaeohyphomycosis fungus.

Herein, the exemplary parasite may include one or more of an intragastrointestinal parasite (for example, a roundworm, hookworm, tapeworm, Entamoeba histolytica, and Giardia lamblia), an intraluminal parasite (for example, Trichomonas vaginalis), an intrahepatic parasite (for example, liver fluke or echinococcus), an intrapulmonary parasite (for example, Paragonimus westermani), a brain tissue parasite (for example, Cysticercus cellulosae or Toxoplasma gondii), an intravascular parasite (for example, schistosome), an intralymphatic parasite (for example, Filariidae), a muscle tissue parasite (for example, Trichinella larva), an intracellular parasite (for example, Plasmodium or Leishmania), a bone tissue parasite (for example, hydatids); a skin parasite (for example, sarcoptic mite or follicular mite), or an intraocular parasite (for example, Thelazia or Cysticercus cellulosae).

According to another aspect of the present invention, the immunostimulatory composition is a tumor therapeutic agent.

In some embodiments, the immunostimulatory composition also includes at least one antibody targeting the tumor antigen.

The definition of the tumor antigen can be the same as above.

The immunostimulatory composition provided in the present invention may further include pharmaceutically acceptable excipients, including, for example, solvents, fillers, buffers, tension modifiers, and preservatives (see, for example, Pramanick et al., Pharma Times, 45:65-77, 2013). In some embodiments, the pharmaceutical composition may include one or more excipients used as solvents, fillers, buffers, and tension modifiers (for example, sodium chloride in saline may be used as an aqueous medium and a tension regulator).

In some embodiments, the immunostimulatory composition further includes one or more of an immune cell therapeutic, a chemical drug, a substance for promoting mucosal immune absorption or mucosal adhesion, an immunomodulator, a ligand for a pattern recognition receptor, and a pharmaceutically acceptable salt or excipient.

The immune cell therapeutic can be one or more selected from tumor-infiltrating lymphocyte, dendritic cell, cytokine-induced killer cell, dendritic cell-cytokine-induced killer cell, natural killer cell, γδT cell, CD3AK, CAR-T and TCR-T.

Examples of the chemical drugs include: alkylation agents such as Thiotepa and cyclophosphamide; sulfonic acid alkyl esters such as busulfan, improsulfan, and piposulfan; aziridines such as benzodopa, carboquone, meturedopa, and uredopa; ethylene imines and methylmelamines, including hexamethylmelamine, triethylenemelamine, triethylenephosphoramide, triethylenethiophosphoramide, and trimethylolmelamine; acetogenin (especially bullatacin and bullatacinone); camptothecin (including synthetic analogs such as topotecan); bryostatin; callystatin; CC-1065 (including its synthetic analogs such as adozelesin and bizelesin); cryptophycins (especially cryptophycin 1 and cryptophycin 8); dolastatin; duocarmycins (including synthetic analogs KW-2189 and CBI-TMI); eleutherobin; pancratistatine; sarcodictyin; spongistatin; chlormethines such as chlorambucil, chlornaphazine, cholophosphamide, estramustine, isophosphamide, chlormethine, chlormethine oxide hydrochloride, Melphalan, novoembichin, fenesterin, prednimustine, trofosfamide, uramustine; nitroureas such as carmustine, chlorozotocin, fotemustine, lomustine, nimustine, and ranimustine; antibiotics such as enediyne antibiotics (for example, calicheamicin, especially calicheamicin γlI and calicheamicin phiI1; see, for example, Agnew, Chem. Intl. Ed. Engl., 33:183-186 (1994); dynemicin, including dynemicin A; bisphosphonate salts such as clodronate salt; esperamicin; and neocarcinostatin chromophore and related chromoprotein enediyne antibiotic chromophore), aclacinomysin, actinomycin, authramycin, azaserine, bleomycin, actinomycin C, carabicin, caminomycin, carzinophilin, chromomycin, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin, and deoxydoxorubicin), epirubicin, esorubicin, idarubicin, marcellomycin, mitomycins such as mitomycin C, mycophenolic acid, nogalamycin, olivomycin, peplomycin, potfiromycin, puromycin, triferricdoxorubicin, rodorubicin, streptonigrin, streptozocin, tubercidin, ubenimex, zinostatin, zorubicin; antimetabolites such as methotrexate and 5-fluorouracil (5-FU); folic acid analogs such as dimethylfolic acid, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, fluorouridine; androgens such as calusterone, drostanolone propionate, epitiostanol, mepitiostane, and testolactone; anti-adrenal drugs such as aminoglutethimide, mitotane, trilostane; folic acid supplements such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; epothilone; etoglucid; gallium nitrate; hydroxyurea; lentinan; lonidamine; maytansines such as maytansine and ansamitocin; mitoguazone; mitoxantrone; mopidamol; nitracrine; pentostatin; phenamet; pirarubicin; losoxantrone; podophyllinic acid; 2-ethylhydrazide; procarbazine; razoxane; rhizoxin; sizofuran; spirogermanium; tenuazonic acid; triaziquone; 2,2′,2″-trichlorotriethylamine; trichothecenes (especially T-2 toxins, verracurin A, roridin A, and anguidine); urethan; vindesine; dacarbazine; mannomustine; dibromomannitol; dibromidulcitol; pipobroman; gacytosine; cytarabine (“Ara-C”); cyclophosphamide; thiotepa; taxanes such as paclitaxel and docetaxel; chlorambucil; gemcitabine; 6-thioguanine; mercaptopurine; methotrexate; platinum analogs such as cisplatin and carboplatin; vinblastine; platinum; etoposide (VP-16); ifosfamide; mitoxantrone; vincristine; vinorelbine; novantrone; teniposide; edatrexate; daunorubicin; aminopterin; Xeloda; ibandronate salt; CPT-11; topoisomerase inhibitor RFS 2000; difluoromethylornithine (DMFO); retinoids such as retinoic acid; capecitabine; and any pharmaceutically acceptable salt, acid, or derivative thereof.

The substance for promoting mucosal immune absorption or mucosal adhesion can be one or more selected from special surfactants, chelating agents, and adhesives, and can be further preferably one or more of polylactic-co-glycolic acid, dextran, and polysaccharides.

The immunomodulator can be one or more selected from chemokine, stem cell growth factor, lymphotoxin, and hematopoietic factor, and can be further preferably one or more of colony-stimulating factor (CSF), interferon, erythropoietin, thrombopoietin, tumor necrosis factor (TNF), interleukin (IL), granulocyte-colony stimulating factor (G-CSF), and granulocyte macrophage-colony stimulating factor (GM-CSF).

The ligands of the pattern recognition receptors can be selected from the ligands of TLR receptors, RLR receptors, CLR receptors, and NLR receptors.

It can be easily understood that the concept of CpG oligonucleotide well-known to persons skilled in the art includes its pharmaceutically acceptable salt form, unless otherwise specified. Exemplary alkaline salts include ammonium salts, alkali metal salts such as sodium, lithium, and potassium salts, alkaline earth metal salts such as calcium and magnesium salts, zinc salts, salts formed with organic alkalis (e.g., organic amines) such as N-Me-D-glucamine, N-[1-(2,3-dioleoyloxy) propyl]-N,N,N-trimethylammonium chloride, choline, trometamol, dicyclohexylamine, and tert-butylamine, and salts formed with amino acids such as arginine and lysine. In addition, the CpG oligonucleotide can be added to a drug solution containing a pharmaceutically acceptable excipient. Alternatively, the CpG oligonucleotide can be provided as lyophilized solid, which can then be reconstituted in sterile water, saline, or a pharmaceutically acceptable buffer before administration.

### Delivery System

Another aspect of the present invention further relates to a delivery system, including: (i) the above CpG oligonucleotide or the above immunostimulatory composition; and (ii) a delivery medium.

In some embodiments, the delivery medium includes one or more liposomes, one or more exosomes, one or more microvesicles, one or more dendrimers, one or more nanocomposites, one or more nanogels, one or more gold nanoparticles, polylactic-co-glycolic acid, one or more cell-penetrating peptides, and a combination thereof.

The liposomes can be cationic liposomes or neutral liposomes, and can be prepared or modified in well-known methods. For example, adding polyethylene glycol (PEG)-modified liposomes can effectively prevent aggregation of liposome vectors and increase their stability. Liposomes or lipid transfection preparations can be prepared in methods known to persons skilled in the art. Such methods are described in, for example, WO 2016205764 and U.S. Pat. Nos. 5,593,972, 5,589,466, and 5,580,859, with all the documents incorporated herein by reference in their entireties.

Dendritic polymers are a special family of polymers that have definite molecular structures and whose chemical structures and unique multivalent characteristics can be precisely controlled, and have gradually become non-viral vectors for gene delivery. Typical dendritic polymers such as poly(amidoamine) (PAMAM) dendritic polymers can be further modified. For example, the surface of PAMAM is modified with a nucleobase analog 2-amino-6-chloropurine to construct a derivative AP-PAMAM, or chondroitin sulfate (CS) is conjugated with PAMAM to prepare CS-PAMAM, and so on.

An example of a preferred nanocomposite is a nanocomposite prepared with CpG and polyethyleneimine (PEI) via electrostatic interaction.

An example of a preferred nanogel is polyethylene glycol (PEG)-based nanogel.

The delivery system can usually improve stability of the CpG and enhance endocytic function of cells, and generally enhance immune activation of the CpG.

### Therapy and Use

The present invention further relates to use of the above CpG oligonucleotide, the above immunostimulatory composition, or the above delivery system in preparation of a medicant for regulating immune cell activity, where the use is conducted in vivo or in vitro.

In some embodiments, the immune cells are selected from macrophages, lymphocytes, and dendritic cells.

In some embodiments, the immune cells are present in human PBMCs.

In some embodiments, the regulation of immune cell activity is to stimulate the immune cells to release inflammatory factors.

In some embodiments, the inflammatory factor includes at least one of IFN-α, TNF-α, and IL-6.

The present invention further relates to use of the above CpG oligonucleotide, the above immunostimulatory composition, or the above delivery system in preparation of a medicant for treating and/or preventing at least one indication, selected from a tumor, a viral infection, a bacterial infection, a fungal infection, a parasitic infection, a chemotherapy side effect, fatigue, or immunosuppression, as well as a low immune response of the subject to an antigen, in a subject in need thereof.

In some embodiments, the indication is related to TLR9-mediated immune response.

In some embodiments, the medicant is in a dosage form for injection, a dosage form for a respiratory route, a nasal drop, a dosage form for skin administration, a dosage form for a mucosal administration, or a dosage form for an intracavitary administration route.

In some embodiments, the antigen includes a tumor, viral, bacterial, fungal, or parasitic antigen.

In some embodiments, the medicant is a vaccine adjuvant or a vaccine.

In some embodiments, the medicant is a vaccine adjuvant or vaccine for a coronavirus, a herpes virus, a hepatitis virus, or an influenza virus.

In some embodiments, the medicant is a vaccine adjuvant or vaccine for a coronavirus, a herpes virus, a hepatitis virus, an influenza A virus, or an influenza B virus. Specifically, the medicant is a vaccine adjuvant or vaccine for type A1 influenza virus, type A3 influenza virus, type Bv influenza virus, or type By influenza virus, or a quadrivalent vaccine adjuvant or vaccine for type A1 influenza virus, type A3 influenza virus antigen, type Bv influenza virus, and type By influenza virus.

In some embodiments, the subject is a mammal.

In some embodiments, the subject is a primate.

In some embodiments, the subject is a human.

The present invention further relates to use of the above CpG oligonucleotide as TLR9 agonist.

The present invention further provides a method for inducing a TLR9-mediated immune response in a subject, comprising administering an effective amount of the above CpG oligonucleotide, or the above immunostimulatory composition to the subject.

The term “effective amount” in the present invention refers to an amount of the ingredient corresponding to the term sufficient to treat, prevent, alleviate and/or relieve the disease or symptom in the subject in the present invention.

In some embodiments, the administration is performed via an intravenous route, an intramuscular route, an intramammary route, an intradermal route, an intraperitoneal route, or a subcutaneous route, by spraying, via an aerosol, via an intra-ovular route, a mucous route, or a transdermal route, by immersion, or via an oral route, an intraocular route, an intratracheal route, or an intranasal route.

In some embodiments, the administration is performed through the above delivery system.

A single dose or multiple doses can be delivered or administered.

In some embodiments, the disease related to TLR9-mediated immune response is at least one indication, selected from a tumor, a viral infection, a bacterial infection, a fungal infection, a parasitic infection, a chemotherapy side effect, fatigue, or immunosuppression, as well as a low immune response of the subject to an antigen.

In some embodiments, the above CpG oligonucleotide, the above immunostimulatory composition, or the above delivery system is used as a vaccine adjuvant or vaccine.

In some embodiments, the above CpG oligonucleotide, the above immunostimulatory composition, or the above delivery system is a vaccine adjuvant or vaccine for a coronavirus, a herpes virus, a hepatitis virus, or an influenza virus.

In some embodiments, the above CpG oligonucleotide, the above immunostimulatory composition, or the above delivery system is a vaccine adjuvant or vaccine for a novel coronavirus, a varicella-zoster virus, a hepatitis B virus, influenza A virus, or influenza B virus. Specifically, the medicant is a vaccine adjuvant or vaccine for type A1 influenza virus, type A3 influenza virus, type Bv influenza virus, or type By influenza virus, or a quadrivalent vaccine adjuvant or vaccine for type A1 influenza virus, type A3 influenza virus antigen, type Bv influenza virus, and type By influenza virus.

In some embodiments, the subject has an infectious disease and is given the above CpG oligonucleotide or the above immunostimulatory composition to elicit an immune response against the pathogen causing the infectious disease.

In some embodiments, the method may be used in combination with the surgery, radiotherapy, chemotherapy, and various immunotherapies, or used in combination with conventional therapies for patients with viral infections, bacterial infections, or parasitic infections.

The embodiments of the present invention are described in detail below with reference to examples. It should be understood that these examples are only used to illustrate the present invention and are not intended to limit the scope of the present invention. In the following examples, for an experimental method for which a specific condition is not specified, preferably, refer to the guidance provided in the present invention, a test manual or a conventional condition in the art, other experimental methods known in the art, or a condition recommended by a manufacturer.

In the following specific examples, a measurement parameter related to an ingredient may have a slight deviation within a weighing accuracy range unless otherwise specified. For temperature and time parameters, an acceptable deviation due to instrument test accuracy or operation accuracy is allowed.

### Example 1 Preparation of Artificially Synthesized CpG-Containing Single-Stranded Oligodeoxynucleotide

The CpG-containing single-stranded oligodeoxynucleotide (CpG-ODN) shown in Table 1 was synthesized by solid-phase synthesis method based on phosphoramidite chemistry.

### 1. Reagents and Materials:

Trichloroacetic acid (TCA), a controllable solid-phase carrier, DMT (dimethoxytrityl), tetrazole activator, acetic anhydride, N-methylimidazole, four types of nucleotide monomers (A, T, C, and G), a DNA synthesizer, a purification instrument, an ultrafiltration instrument, a vacuum dryer, a high-performance liquid chromatograph, and the like.

### 2. Method:

### Deprotection

By using trichloroacetic acid (TCA), the protecting group dimethoxytrityl (DMT) was removed from the nucleotide ligated to the controllable solid-phase carrier (controlled pore glass), to obtain free 5′-hydroxyl end to be used for the next condensation reaction.

### Activation

The phosphoramidite-protected nucleotide monomer and the tetrazole activator were mixed and added into the synthesis column, to form an active intermediate of phosphoramidite-tetrazole (having an activated 3′-end and a DMT-protected 5′-end). This intermediate and the deprotected nucleotide on the controllable solid-phase carrier were subjected to the condensation reaction.

### Ligation

When mixed with a deprotected nucleotide on the controllable solid-phase carrier, the active intermediate of phosphoramidite-tetrazole was subjected to a nucleophilic reaction with a 5′-hydroxyl group of the deprotected nucleotide, to be condensed and deprived of tetrazole, and at this time, the synthesized oligonucleotide chain had one more base extending forward.

### Blocking

After the condensation reaction, to prevent the unreacted 5′-hydroxyl group ligated to the controllable solid-phase carrier from being extended in subsequent circular reactions, the terminal hydroxyl group was often blocked by acetylation, and usually an acetylation agent was usually prepared by mixing acetic anhydride, N-methylimidazole, and the like.

### Oxidation

Through phosphite ester linkages, during the condensation reaction, the nucleotide monomer was ligated to the oligonucleotide ligated to the controllable solid-phase carrier. However, the phosphite ester linkages were unstable and likely to be hydrolyzed by acids and bases. At this time, a solution of iodine in tetrahydrofuran was commonly used to convert the phosphite into a phosphotriester, to obtain stable oligonucleotides.

After the above five steps, one deoxynucleotide was ligated to the nucleotide on the controlled solid-phase carrier. Similarly, trichloroacetic acid was further used to remove the protecting group DMT from the 5′-hydroxyl group of the newly ligated deoxynucleotide, and the processes of activation, ligation, blocking, and oxidation were repeated to obtain a crude DNA fragment. Finally, the crude DNA fragment was subjected to post-synthesis treatments such as cutting, deprotection (commonly, A and C bases were protected by the benzoyl group; G base was protected by the isobutyryl group; T base needed no protection; and phosphorous acid was protected by the cyanoethyl group), purification (methods such as HAP, PAGE, HPLC, C18, and OPC were commonly used), and quantification to obtain oligonucleotide fragments that meet experimental requirements.

Unsulfurized CpG single-stranded deoxynucleotides were synthesized on the ABI 3900 DNA synthesizer; and fully and partially sulfurized CpG single-stranded deoxynucleotides were synthesized in the displacement method on the ABI 394 DNA synthesizer.

TABLE 1
Artificially Synthesized CpG-Containing
Single-Stranded Oligodeoxynucleotide

| SEQ ID | | | Length |
|---|---|---|---|
| NO | Code | Oligonucleotide Sequence | (bp) |
| 1 | CpG1 | 5′-TCGCAACGTTGCCTTCGAAGG-3′ | 21 |
| 2 | CpG2 | 5′-TCGCGTGACGTCATCGATCGATCGGG-3′ | 26 |
| 3 | CpG3 | 5′-TCGTACGTAGGCGCAATCGATTGGGG-3′ | 26 |
| 4 | CpG4 | 5′-TCGATCGCACGTGCCGTCGACGGCG-3′ | 25 |
| 5 | CpG5 | 5′-TCGGGGACGTCAAGTCGACTTGGGG-3′ | 25 |
| 6 | CpG6 | 5′-TCGCGTACGTCGGTCGATCGCGCG-3′ | 24 |
| 7 | CpG7 | 5′-TCGCTACGTGCGGATCGAGCGCCG-3′ | 24 |
| 8 | CpG8 | 5′-TCGACGCACGTGGGCCTCGAGGG-3′ | 23 |
| 9 | CpG9 | 5′-TCGCGTCGGACGTGCGTCGACGG-3′ | 23 |
| 10 | CpG10 | 5′-TCGTACGTACGCCGGTCGACCG-3′ | 22 |
| 11 | CpG11 | 5′-TCGCTACGTGAGTCGACTCGGG-3′ | 22 |
| 12 | CpG12 | 5′-TCGTTGACGTCGCATCGATCG-3′ | 21 |
| 13 | CpG13 | 5′-TCGCACGTGCCGTCGACGCCG-3′ | 21 |
| | CpG14 | 5′-TCGGACGTCGCAGTCGACGG-3′ | 20 |
| 15 | CpG15 | 5′-TCGTACGTACGTCGACGGG-3′ | 19 |
| 16 | CpG | 5′-ggGGGACGATCGTCgggggG-3′ | 20 |
| | 2216 | | |
| 17 | CpG | 5′-TCGTCGTTTTGTCGTTTTGTCGTT-3′ | 24 |
| | 7909 | | |
| 18 | CpG | 5′-TGACTGTGAACGTTCGAGATGA-3′ | 22 |
| | 1018 | | |
| 19 | CpG | 5′-TCGTCGTTTTCGGCGCGCGCCG-3′ | 22 |
| | 2395 | | |
| 20 | FX-700 | 5′-TGGCCAAGCTTGGGCCCCTTGCAAGGGCC-3′ | 29 |

Note:
Capital letters represent a phosphorothioate backbone; and lowercase letters represent a phosphodiester backbone.

### Example 2 Screening for Active CpG

A proliferation effect of CpG on human PBMCs and mouse spleen cells, namely T and B cells, was tested, to screen active sequences in CpG1-15 prepared in Example 1.

Type A CpG 2216, Type B CpG 1018, and Type C CpG 2395 were used as positive control groups, while FX-700 was used as a negative control group. The CpG in Example 2 and all the following examples were prepared by the inventors' company independently.

### 1. Reagents and Materials:

White blood cells concentrated from human peripheral blood, saline (Dubang, China), PBS (BI, Israel), Ficoll-Paque PLUS (GE, USA), RPMI-1640 culture medium (Corning, USA), FBS (Clark, USA), penicillin/streptomycin (TransGen Biotech, China), trypan blue (Sigma, USA), CellTrace™ CFSE Cell Proliferation Kit (InvitroGen, USA), Human TruStain FcX™ (Fc receptor blocking solution) (BD, USA), human CD45-PerCP-Cy5.5 (BD, USA), human CD3-APC (BD, USA), human CD19-V450 (BD, USA); cell filter screen (BD Falcon, USA), red blood cell lysis buffer (BD, USA), LIVE/DEAD™ fixable aqua dead cell stain kit (InvitroGen, USA), mouse CD16/CD32 antibody (2.5G2) (BD, USA), mouse CD45-PerCP-Cy5.5 (30-F11) (BD, USA), mouse CD3e-PE-Cy7 (145-2C11) (BD, USA), and mouse CD19-APC (1D3) (BD, USA); and RPMI-1640 complete medium: RPMI-1640 medium containing 10% fetal bovine serum and 100 U/mL-100 μg/mL Pen-Strep (penicillin-streptomycin).

Red blood cell lysis buffer: 10× of red blood cell lysis buffer was diluted with sterile water to 1×, that is, 4500 μL of sterile water was added to 500 μL of red blood cell lysis buffer.

Solution of carboxyfluorescein diacetate succinimidyl ester (CFSE): CFSE was dissolved with DMSO to prepare a 1 mg/mL stock solution, and when being used, the stock solution was diluted with serum-free PBS until a working concentration reached 5 μg/mL.

### 2. Method:

Isolation of mouse spleen cells: BALB/c mice were immersed in 75% ethanol immediately after euthanasia. In the super clean bench, a small opening was cut on the left side of the mouse's abdomen, and the mouse spleen was ripped off with forceps and placed in a Petri dish containing a small amount of prechilled RPMI-1640 medium. The spleen was gently ground into a chylous fluid with a plunger rod of a 1 ml syringe. The cells were filtered with a 100 μm filter screen. The single cell suspension of the mouse spleen was collected and centrifuged at 1500 rpm for 5 minutes. The supernatant was discarded, and the cells were resuspended with 2-3 mL of red blood cell lysis buffer and incubated on ice for 2 minutes. 15-20 mL of RPMI-1640 complete medium was added to stop the reaction and the resulting mixture was centrifuged at 1500 rpm for 5 minutes. The supernatant was discarded, mouse spleen cells were resuspended in PBS, and the Trypan blue staining method was used for cell counting. 10 mL of PBS was added, and the resulting solution was centrifuged at 1500 rpm for 5 minutes.

Isolation of hPBMC: White blood cells concentrated from human peripheral blood were 3-fold diluted with normal saline. The diluted white blood cells concentrated from human peripheral blood were slowly added to the upper layer of Ficoll-Paque PLUS at a ratio of 1:1, the rotation speed was risen by the 8th gear and decreased by the 1st gear, and the resulting mixture was centrifuged at 2500 rpm for 25 minutes. The white film layer was extracted, 20 mL of normal saline was added, and the resulting solution was centrifuged at 1800 rpm for 10 minutes. The supernatant was discarded, 20 mL of normal saline was added to resuspend the cells, and the resulting solution was centrifuged at 1500 rpm for 8 minutes. The supernatant was discarded, 20 mL of normal saline was added to resuspend the cells, and the resulting solution was centrifuged at 1500 rpm for 5 minutes. The supernatant was discarded, the mononuclear cells were resuspended in PBS, and the Trypan blue staining method was used for cell counting. PBS was added to dilute the solution to 45 mL, and the resulting solution was centrifuged at 1500 rpm for 5 minutes.

Labeling of Cells with CFSE: Human PBMCs or mouse spleen cells were resuspended with the PBS until the density reached 1×10⁷ cells/mL. 5 μL of CFSE was added to every 1 mL of the resuspension for staining. The resuspension was incubated at room temperature in the dark for 7 minutes (human PBMCs) or 5 minutes (mouse spleen cells). Prechilled RPMI-1640 complete medium was added to stop the reaction and the resulting mixture was centrifuged at 1500 rpm for 5 minutes. 10-20 mL of PBS was added twice for rinsing and the resulting mixture was centrifuged at 1500 rpm for 5 minutes. The supernatant was discarded, and the cells were resuspended with RPMI-1640 complete medium until the density reached 5×10⁶ cells/mL.

CpG ODN Stimulation: 100 μL of the prepared cell suspension (about 5×10⁵ cells) was added to each well of a 96-well U-shaped plate, then 100 μL of CpG 1-15, CpG 2216, CpG 1018, CpG 2395, and FX-700 solutions were added to each well of the 96-well U-shaped plate, so that the final concentration of the CpG was 1 μM. In addition, unstimulated wells containing only cells and culture medium (stimulation concentration was 0) was arranged. The culture plates were incubated in an incubator containing 5% CO₂ at 37° C. and saturated humidity for 5 days (for hPBMCs) or 3 days (for mouse spleen cells).

Detection of Proliferation of T and B Cells via Flow Cytometry: The cells were collected into a flow cytometry tube and centrifuged at 1500 rpm for 5 minutes. The supernatant was discarded, and 2 mL of PBS was added to resuspend the cells. 2 μL of Human TruStain FcX™ (for hPBMCs) or mouse CD16/CD32 antibody (for mouse spleen cells) were added and the cells were incubated for 10 minutes at room temperature in the dark for non-specific blocking. 0.5 μL of Aqua was added to distinguish between dead and live cells. Anti-CD45, anti-CD3, and anti-CD19 mAbs were added for cell surface staining, and mixed well, and the cells were incubated at 4° C. for 25-30 minutes. The cells were rinsed with 1-2 mL of PBS twice and the resulting mixture was centrifuged at 1500 rpm for 5 minutes. The supernatant was discarded, the cells were resuspended with 300 μL of PBS, and then the proliferation of T and B cells was detected by LSRFortessa™ Flow Cytometry (BD, USA).

### 3. Results:

For the results, refer to Table 2.

TABLE 2
CpG-Induced Proliferation of Human and Mouse B Cells

| | Human | Mouse |
|---|---|---|
| B Cells | Cell proliferation | Cell proliferation |
| CpG1 | + | + |
| CpG 2 | − | − |
| CpG 3 | − | − |
| CpG4 | − | − |
| CpG5 | − | − |
| CpG6 | − | − |
| CpG7 | − | − |
| CpG8 | − | − |
| CpG9 | − | − |
| CpG10 | − | − |
| CpG11 | − | − |
| CpG12 | − | − |
| CpG13 | − | − |
| CpG14 | − | − |
| CpG15 | − | − |
| CpG 2216 | − | − |
| CpG 1018 | + | − |
| CpG 2395 | + | − |

It can be seen from the results in Table 2 that only CpG1 in CpG1-15 has the activity of stimulating proliferation of B cells, and is effective on both human PBMC and mouse spleen cells. In addition, all CpGs have no significant stimulation effect on the proliferation of T cells in human PBMCs and mouse spleen cells.

### Example 3 Testing of Biological Activity of CpG ODN Via HEK Blue mTLR9 Cells

The levels of the secreted embryonic alkaline phosphatase (SEAP) of the HEK-Blue™ mTLR9 cell line were measured to evaluate activation effects of different CpGs at different concentrations on mouse TLR9. HEK-Blue™ mTLR9 cells (expressing murine TLR9) were stimulated with CpG1, CpG 2395, CpG 7909, and CpG 1018, and FX-700 (non-CpG) in the negative control group at certain concentrations, respectively, to activate NF-κB and AP-1, thereby inducing production of SEAP. The SEAP could be detected by Quanti-Blue™ reagent, which appeared blue when bound to the SEAP, and the level of the SEAP was quantified by measuring the OD value at 630 nm via a spectrophotometer to assess the activation of mouse TLR9 by different CpGs.

### 1. Reagents and Materials:

HEK-Blue™ mTLR9 cells (WCB cells); and

DMEM (Gibco, 11995-065); FBS (Gibco, 10099-141C); Zeocin™ (ant-zn-05, InvivoGen, USA); Blasticidin (ant-b1-05, InvivoGen, USA); Normocin™ (ant-nr-1, InvivoGen, USA), Pen-Strep (Gibco, 15140-122); Quanti-Blue (Invivogen, rep-qbl); and 96-well plate (Costar, 3599).

Growth medium: DMEM medium containing 4.5 g/L glucose, 2 mM L-glutamine, 10% heat-inactivated fetal bovine serum, 100 μg/mL Normocin, and Pen-Strep (100 U/mL-100 μg/mL). After the thawed cells were subcultured twice, 100 μg/mL Zeocin and 30 μg/mL Blasticidin should be supplemented in the growth medium as selective antibiotics.

QUANTI-Blue™ detection solution: 1 mL of QB reagent and 1 mL of QB buffer were added to a 250 mL sterile culture flask, then sterile water was added until a total volume reached 100 mL and the resulting mixture was mixed uniformly by vortex mixing. The resulting mixture was used after 10 minutes of incubation at room temperature.

Test sample solution: Appropriate amounts of the test samples of lyophilized powder (CpG1, CpG 2395, CpG 7909, and CpG 1018, and FX-700 in a negative control group) were weighed, dissolved in sterile PBS, and then diluted to the required concentration with the growth medium that was not supplemented with the selective antibiotics.

### 2. Method:

In a 37° C. incubator containing 5% CO₂, the HEK-Blue mTLR9 cell line was cultured in vitro as monolayer adherent cells in growth medium supplemented with the selective antibiotics. The cell culture medium was exchanged twice a week, and typically the cells were subcultured by no more than 15 generations at a density of at least 0.5×10⁶ cells/mL (the cell density should not exceed 6×10⁶ cells/mL) within one week.

HEK-Blue™ mTLR9 cells at the logarithmic phase were selected, washed and digested to prepare single cells, the cells were resuspended in fresh growth medium supplemented with the selective antibiotics, and the cell density was adjusted to 2-3×10⁵ cells/mL.

180 μL of the prepared cell suspension was added to each well in a U-shaped 96-well plate. The culture plates were incubated in a 37° C. incubator containing 5% CO₂ at saturated humidity for about 1-24 hours, and then each well was supplemented with the liquid until a volume of 180 μL. 20 μL of the test sample solutions at different concentrations was added to the corresponding wells of the U-shaped 96-well plate, and final concentrations were 0, 0.25, 0.5, 1, 2, 5, 10, 20, and 40 μM. Triplicate wells were arranged for each concentration and the cells were further cultured for 24 hours.

After culture, the U-shaped 96-well plate was centrifuged at 1000 rpm for 5 minutes, 40 μL of supernatant was aspirated from each well and placed into a flat-bottomed 96-well plate. 160 μL of QUANTI-Blue™ test solution was quickly added, and the resulting mixture was oscillated briefly and further incubated in a 37° C. incubator containing 5% CO₂ for 15 minutes. The 96-well test plate was taken out and the OD value was measured at a wavelength of 630 nm by using a spectrophotometer.

### 3. Results:

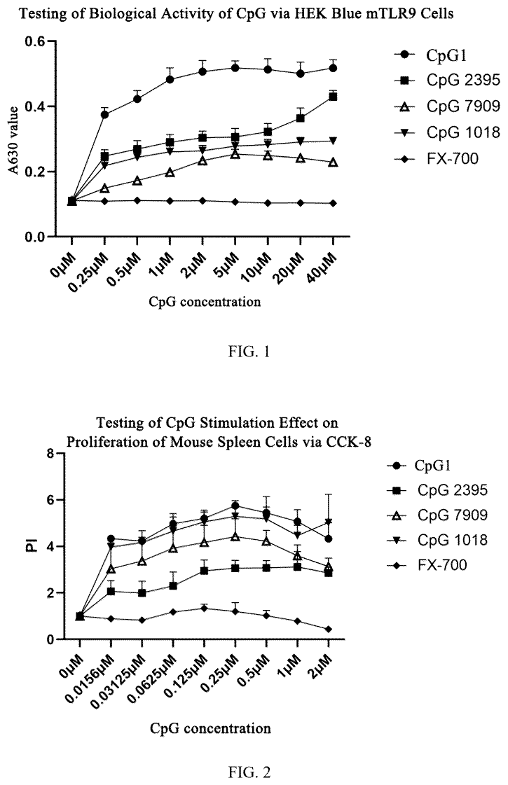

Referring to FIG. 1, FIG. 1 indicates that each sequence has a significant response, which is positively correlated with the dosage. The response to CpG1 is relatively strong, followed by the response to CpG 2395, CpG 1018, and CpG 7909, while the reverse sequence FX-700 has no activating effect on mTLR9.

### Example 4 Testing of CpG ODN Stimulation Effect on Proliferation of Mouse Spleen Cells Via CCK-8

The effects of different CpGs on the proliferation of mouse splenic lymphocytes were tested with the CCK-8 (Cell Counting Kit-8) method by using a multifunctional microplate reader. The detection principle is as follows: WST-8 (2-(2-methoxy-4-nitrophenyl)-3-(4-nitrophenyl)-5-(2,4-disulfophenyl)-2H-tetrazolium monosodium salt) can be reduced by some dehydrogenases in mitochondria to produce orange-yellow water-soluble formazan in the presence of the electron coupling reagent 1-methoxy PMS. The color depth of the generated formazan is proportional to cell proliferation and inversely proportional to cell toxicity. The OD value at the wavelength of 450 nm was measured via a microplate reader, which indirectly reflected the number of viable cells.

### 1. Reagents and Materials:

96-well plate (Costar, 3599);
CCK-8 (Dojindo Laboratories, CK04); FBS (Gibco, 10099-141C); Pen-Strep (Gibco, 15140-122); and RPMI-1640 (Sigma, R8758-500 mL); and
RPMI-1640 complete medium: RPMI-1640 medium containing 10% fetal bovine serum and 100 U/mL-100 μg/mL Pen-Strep (penicillin-streptomycin).

### 2. Method:

Isolation of mouse spleen cells: The mice were euthanized by cervical dislocation and then completely immersed in a container containing 75% alcohol for 3 minutes. The mouse was fixed in a biosafety cabinet, the abdominal cavity was opened, the fat and fascial tissues were cut off, the spleen was taken out, and the blood was washed off with normal saline. An appropriate amount of normal saline was added to one petri dish, a 100 μm cell strainer was placed, and the spleen was taken and put into the cell strainer. A nozzle of a clean 10 mL or 20 ml syringe was taken and used to crush the tissue via its end for pressing. The cells inside the membrane were dissociated gradually and after being passed through the cell strainer, the cells were suspended in the solution in the petri dish, and rinsed with normal saline until the tissue became light in color. The cell strainer with 70 μm pore size was put on a 50 mL centrifuge tube and the 70 μm strainer was rinsed with 2 mL of normal saline. The cells were aspirated into the 70 μm strainer and filtered, and after filtration, the strainer was rinsed with 2 mL of normal saline twice, and all the cell suspension was collected into a 50 mL centrifuge tube. The resulting mixture was centrifuged at 319 g for 5 minutes at room temperature, the supernatant was discarded, and the cell precipitate was collected. Prechilled 1× red blood cell lysis buffer was added to the cell precipitate, the cells were gently dispersed by blowing, and incubated on ice while being manually inverted and shaken several times every other 1 minute, and after 5 minutes, 20 mL of normal saline was added. The mixture was centrifuged at 319 g for 5 minutes. After centrifugation, the supernatant was discarded, and the cell precipitate was collected.

Cell Inoculation: The mouse spleen cells were resuspended with the complete culture medium, the cell density was adjusted to 2×10⁷ cells/mL according to the cell counting results, and 100 μL of the resulting mixture was added into each well of the 96-well plate and incubated in a 37° C. incubator containing 5% CO₂ for approximately 1-24 hours.

CpG ODN Stimulation: The test samples (CpG1, CpG 2395, CpG 7909, and CpG 1018, FX-700 as a negative control) were diluted with the complete culture medium, 10 μL of the resulting mixture was added to each well of the 96-well plate, and triplicate wells were arranged for each concentration. In the experiment, 3 control wells containing cells but no CpG ODN and 3 blank wells containing no cells or CpG were arranged in each cell plate. The working concentrations of CpG ODNs were sequentially 0, 0.0156, 0.03125, 0.0625, 0.125, 0.25, 0.5, 1, and 2 μM. After mixing, the resulting mixture was put in a 37° C. incubator containing 5% CO₂ and incubated for about 72 hours.

CCK-8 Testing: 10 μL of CCK-8 solution was added to each well, the culture plate was incubated in an incubator for 1-4 hours, and the absorbance was measured at 450 nm by using a microplate reader.

### 3. Results

Referring to FIG. 2, as shown in FIG. 2, FX-700 cannot induce the proliferation of mouse spleen cells, and with the increase in FX-700 concentration, the GC inhibits the proliferation of the mouse spleen cells. In addition to FX-700, all other tested CpG ODNs showed the capability to induce the proliferation of the mouse spleen cells. In addition, most CpG ODNs at 0.25 μM showed the peak induction capability. A descending sequence of the proliferation induction capacities of various CpG ODNs at a concentration of 0.25 μM is as follows: CpG1>CpG 1018>CpG 7909>CpG 2395.

### Example 5 Cytokine Activity of Human PBMCs after Stimulation with Different CpGs

After stimulation of normal human PBMCs in vitro with different CpGs at different concentrations for 16-24 hours, the levels of cytokines such as IFN-α, TNF-α, and IL-6 in the cell supernatant were measured to evaluate immunostimulating activity.

### 1. Reagents and Materials:

RPMI-1640 (Gibco, 11875-093); FBS (Gibco, 10099-141C); Pen-Strep (Gibco, 15140-122); Ficoll-Paque™ PLUS (GE, 17144003-1); Human TNF-α Precoated ELISA kit (Dayou, 1117202); Human IFN-α Precoated ELISA kit (Dayou, 1110012); and Human IL-6 Precoated ELISA kit (Dayou, 1110602);
fresh whole blood of healthy human; and
complete medium: RPMI-1640 medium containing 10% fetal bovine serum and 100 U/mL-100 μg/mL Pen-Strep.

### 2. Method:

Isolation of Human PBMC: 30 mL of peripheral blood was transferred into a 100 mL sterile flask, and 30 mL of normal saline was added at a ratio of 1:1, and mixed well. Ten 15 mL sterile centrifuge tubes were taken, 5 mL of Ficoll-Paque™ PLUS was added to each centrifuge tube, and then 6 mL of diluted blood was slowly added, without damaging the interface. The mixture was transferred to the centrifuge and centrifuged at 960 g at room temperature for 30 minutes. The white film layer was aspirated and added to a 50 mL centrifuge tube, 20 mL of normal saline was added, and the resulting solution was centrifuged at 319 g at room temperature for 10 minutes. The supernatant was discarded, 40 mL of normal saline was added to resuspend the cells, and the resulting solution was centrifuged at 460 g at room temperature for 5 minutes. The cells were resuspended in 5 mL of complete culture medium, and the suspension concentration was adjusted to 1-2×10⁶/mL for later use according to the cell count.

Cell Inoculation: According to the layout design of the pore plate, 1 mL of the cell solution with adjusted concentration was added to each well of a 96-well deep well plate, and incubated in a 37° C. incubator containing 5% CO₂ for at least 1 hour.

CpG ODN Stimulation: The CpG ODN was diluted with the complete medium, and 10 μL of the resulting mixture was added to each well of the above 96-well deep well plate, with no duplicates for a single concentration. The working concentrations of CpG ODNs were 0, 0.0156, 0.03125, 0.0625, 0.125, 0.25, 0.5, 1, 2, 5, and 10 μM. The resulting mixture was put in a 37° C. incubator containing 5% CO₂ and incubated for about 16-24 hours.

Detection of Cytokine Levels with ELISA Method: Contents of IFN-α, IL-6, and TNF-α in the cell supernatant were measured with the detection method provided in the leaflet of each kit.

### 3. Results:

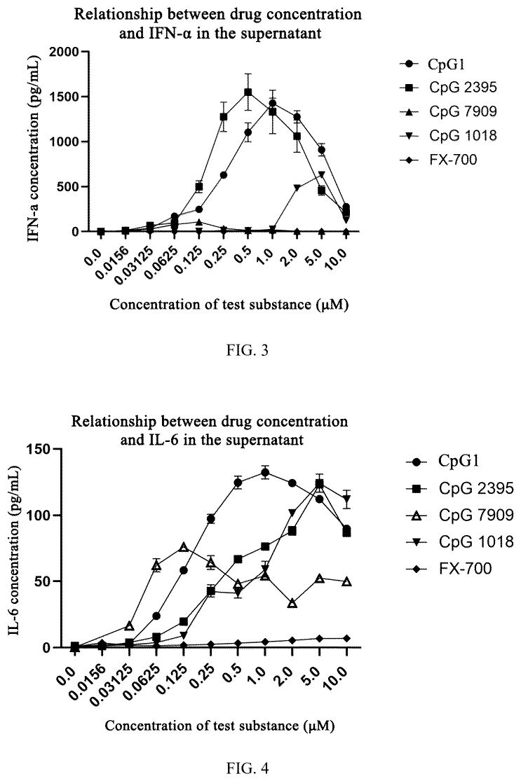

Referring to FIG. 3 to FIG. 5, as shown in FIG. 3, CpG1 and CpG 2395 have equivalent capabilities to stimulate human PBMCs to secret IFN-α, demonstrating strong stimulation effects that are significantly better than those of CpG 1018 and CpG 7909; as shown in FIG. 4, when the concentration of CpG ODN is between 0.25-2 μM, CpG1 significantly outperforms CpG 1018, CpG 2395, and CpG 7909 in stimulating human PBMCs to secret IL-6; and as shown in FIG. 5, all the CpG ODNs have a certain capability to stimulate human PBMCs to secret TNF-α compared to the negative control group.

### Example 6 Effect of CpG on Immunogenicity of Novel Coronavirus Vaccine Composition

Titer of anti-BA 4/5 antibody in the serum of mice after immunization was tested to evaluate the effect of CpG1 on the immunogenicity of the novel coronavirus recombinant subunit vaccine composition.

### 1. Reagents and Materials:

Six- to eight-week-old female BALB/c mice, purchased from Beijing Vital River Laboratory Animal Technology Co., Ltd.;
normal saline, purchased from Qingdao Hi-Tech Industrial Park Haiao Bio-Technology Co., Ltd.;
antigen Omicron BA.4/BA.5 RBD protein (hereinafter referred to as BA 4/5), with the category number of SPD-C522r, purchased from ACRO; and CpG 1, obtained as previously described in Example 2; and an adjuvant of aluminum hydroxide (Sinopharm).

### 2. Method:

A novel coronavirus vaccine composition was prepared by using normal saline as the solvent. Each 200 μL of the vaccine composition contained 5 μg of BA 4/5, 90 μg of Al(OH)₃ (counted as aluminum), and 1 μg of CpG 1 (if present), with the remainder being normal saline.

The mice were randomly grouped, each group contained 24 mice, and the mice were immunized according to Table 3. Each group of BALB/c mice was immunized with 200 μL of intramuscular injection on days 0 and 28, separately, with the first dose being administered on day 0 and a group immunized with the normal saline as a control group. Blood samples were taken on D7, D29, and D56, with 12 blood samples collected in each group. On D7, blood was sampled from the retro-orbital venous plexus, while on D29 and D56, blood was sampled by eyeball enucleation.

TABLE 3
Grouping of Mice

| | | | | Content of | |
|---|---|---|---|---|---|
| | | Antigen | CpG | aluminum | Number |
| Groups and Names | | (μg/dose) | (μg/dose) | (μg/dose) | of mice |
| 1 | Blank control group | — | — | — | 24 |
| | (normal saline) | | | | |
| 2 | BA 4/5 + aluminum | 5 | — | 90 | 24 |
| | hydroxide | | | | |
| 3 | BA 4/5 + aluminum | 5 | 1 | 90 | 24 |
| | hydroxide + CpG 1 | | | | |

Mouse blood was sampled at specified time points, serum was obtained by centrifugation, and the titer of the anti-BA 4/5 antibody was measured by indirect ELISA (coated antigen was Omicron BA.4/BA.5 RBD protein).

### 3. Results:

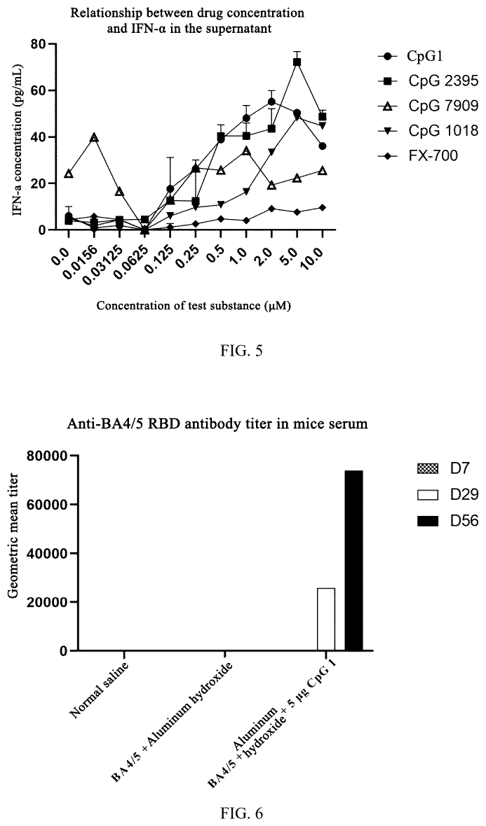

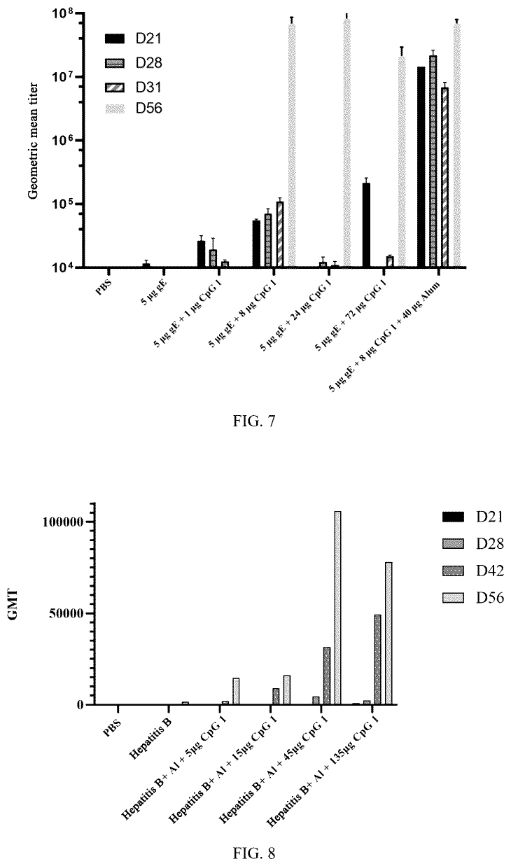

Referring to FIG. 6, it can be learned from FIG. 6 that neither the normal saline group nor the group treated with the single adjuvant of aluminum hydroxide exhibits immunogenicity. The dual-adjuvant group shows higher antibody titers. The antibody titers are positively correlated with the number of days. The dual-adjuvant group can maintain a high antibody titer on D56. On D56, the antibody titer of the group treated with BA4/5, aluminum hydroxide, and CpG 1 is the highest and is 2-5 times that of the group treated with the same dose.

### Example 7 Effect of CpG on Immunogenicity of Varicella-Zoster Virus Vaccine Composition in Mice

A titer of gE protein-specific antibody in the serum of mice after immunization was detected to evaluate the effect of CpG1 on the immunogenicity of the varicella-zoster virus vaccine composition.

### 1. Reagents and Materials:

female C57BL/6J mice, purchased from Beijing Vital River Laboratory Animal Technology Co., Ltd.;
dry powder of PBS phosphate buffer, purchased from Solarbio;
gE protein, produced by the inventors and having a sequence as shown in SEQ ID NO: 21

(MGTVNKPVVGVLMGFGIITGTLRITNPVRASVLRYDDFHIDEDK
LDTNSVYEPYYHSDHAESSWVNRGESSRKAYDHNSPYIWPRNDYD
GFLENAHEHHGVYNQGRGIDSGERLMQPTQMSAQEDLGDDTGIHV
IPTLNGDDRHKIVNVDQRQYGDVFKGDLNPKPQGQRLIEVSVEEN
HPFTLRAPIQRIYGVRYTETWSFLPSLTCTGDAAPAIQHICLKHT
TCFQDVVVDVDCAENTKEDQLAEISYRFQGKKEADQPWIVVNTST
LFDELELDPPEIEPGVLKVLRTEKQYLGVYIWNMRGSDGTSTYAT
FLVTWKGDEKTRNPTPAVTPQPRGAEFHMWNYHSHVFSVGDTFSL
AMHLQYKIHEAPFDLLLEWLYVPIDPTCQPMRLYSTCLYHPNAPQ
CLSHMNSGCTFTSPHLAQRVASTVYQNCEHADNYTAYCLGISHME
PSFGLILHDGGTTLKFVDTPESLSGLYVFVVYFNGHVEAVAYTVV
STVDHFVNAIEERGFPPTAGQPPATTKPKEITPVNPGTSPLIRYA
AWTGGLA);

and

CpG 1, obtained as previously described in Example 2; and an adjuvant of aluminum hydroxide (Alum) (Sinopharm).

### 2. Method:

A varicella-zoster virus vaccine composition was prepared by using PBS solution as the solvent. Each 50 μL of the vaccine composition contained 5 μg of gE protein, doses of Alum and CpG 1 (if present) in each group are shown in Table 4, with the remainder being PBS solution.

The eight-week-old female C57BL/6J mice were randomly grouped, each group contained 12 mice, and the mice were immunized according to Table 4. Each group of mice was immunized with 50 μL of intramuscular injection on days 0 and 28, separately, with the first dose being administered on day 0 and a group immunized with the PBS solution as a control group. Blood was sampled from the orbits on D21, D28 (before the second immunization), D31, and D56, and 200-300 μL of blood was sampled from each mouse each time. Serum was isolated and the titer of binding antibody was measured with the ELISA method, and the antibody titer was expressed as the geometric mean titer (GMT). Grouping of the mice is shown in Table 4.

TABLE 4
Grouping of Mice

| Group | Composition and dosage of vaccines |
|---|---|
| 1 | PBS |
| 2 | 5 μg of gE |
| 3 | 5 μg of gE + 1 μg of CpG 1 |
| 4 | 5 μg of gE + 8 μg of CpG 1 |
| 5 | 5 μg of gE + 24 μg of CpG 1 |
| 6 | 5 μg of gE + 72 μg of CpG 1 |
| 7 | 5 μg of gE + 8 μg of CpG 1 + 40 μg of Alum |

### 3. Results:

Referring to FIG. 7, it can be learned from FIG. 7 that CpG 1 can enhance the immunogenicity of the antigen, and as the dose of CpG increases within the dosage range of 1-72 μg, the immunity enhancement effect of CpG 1 shows a trend of initially increasing and then decreasing. In addition, compared with the vaccine group containing single adjuvant of CpG 1, the vaccine group containing dual adjuvants of CpG 1 and Alum significantly enhances the titer of the antibody specific to VZV gE (by more than 100 times).

### Example 8 Effect of CpG on Immunogenicity of Hepatitis B Vaccine Composition in Mice

The level of anti-HBs antibody in the serum of mice after immunization was detected to evaluate the effect of CpG1 on the immunogenicity of the hepatitis B vaccine composition.

### 1. Reagents and Materials:

female BALB/c mice, purchased from Beijing Vital River Laboratory Animal Technology Co., Ltd.;
dry powder of PBS phosphate buffer (Solarbio) and hepatitis B stock solution; (Walvax) and
CpG 1, obtained as previously described in Example 2; and an adjuvant of aluminum hydroxide (Sinopharm).

### 2. Method:

A hepatitis B vaccine composition was prepared by using PBS solution as the solvent. Each 1 mL of the vaccine composition contained 1 μg of antigen, 0.43 mg of aluminum hydroxide (calculated as per aluminum), and 5, 15, 45, or 135 μg of CpG 1 (if present), with the remainder being the PBS solution.

The six- to eight-week-old female BALB/c mice were randomly grouped, each group contained 10 mice, and the mice were immunized according to Table 5. Each group of mice was immunized with 1 mL of intraperitoneal injection on days 0 and 28, separately, with the first dose being administered on day 0 and a group immunized with the PBS solution as a control group. Blood was sampled on D21, D28 (before the second immunization), D42, and D56, and 200-300 μL of blood was sampled from each mouse each time. Serum was isolated and anti-HBs antibody levels in serum samples were detected with a commercially available hepatitis B surface antibody kit, and the antibody titer was expressed as the geometric mean titer (GMT). Grouping of the mice is shown in Table 5.)

TABLE 5
Grouping of Mice

| | | Antigen | CpG | Content of | Number |
|---|---|---|---|---|---|
| | | (μg/ | (μg/ | aluminum | of |
| Groups and Names | | dose) | dose) | (mg/dose) | mice |
| 1 | PBS | — | — | — | 10 |
| 2 | Hepatitis B | 1 | — | — | 10 |
| 3 | Hepatitis B + aluminum | 1 | 5 | 0.43 | 10 |
| | hydroxide + CpG 1 (low) | | | | |
| 4 | Hepatitis B + aluminum | 1 | 15 | 0.43 | 10 |
| | hydroxide + CpG 1 | | | | |
| | (medium) | | | | |
| 5 | Hepatitis B + aluminum | 1 | 45 | 0.43 | 10 |
| | hydroxide + CpG 1 (high) | | | | |
| 6 | Hepatitis B + aluminum | 1 | 135 | 0.43 | 10 |
| | hydroxide + CpG 1(highest) | | | | |

Note:
Hepatitis B in this table refers to the hepatitis B stock solution containing the hepatitis B antigen.

### 3. Results:

Referring to FIG. 8, It can be seen from FIG. 8 that CpG1 can enhance the immunogenicity of the antigen in a dose-dependent manner within the dosage range of 5-45 μg per dose, and the immunogenicity of the vaccine composition decreases when the dosage of CpG 1 is further increased to 135 μg.

### Example 9 Effect of CpG on Immunogenicity of Influenza Vaccine Composition in Mice

The levels of anti-A1 antigen antibody, anti-A3 antigen antibody, anti-Bv antigen antibody, and anti-By antigen antibody in the serum of mice after immunization were detected to evaluate the effect of CpG1 on the immunogenicity of the quadrivalent influenza vaccine composition.

### 1. Reagents and Materials:

female BALB/c mice, purchased from Beijing Vital River Laboratory Animal Technology Co., Ltd.;
dry powder of PBS phosphate buffer (Solarbio);
type-A1, type-A3, type-By, and type-Bv influenza antigens, (SinoBiological); and
CpG 1, obtained as previously described in Example 2.

### 2. Method:

A quadrivalent influenza vaccine composition was prepared by using PBS solution as the solvent. Each 0.1 mL of the vaccine composition contained 3.5 μg of each antigen (A1, A3, Bv, and By) and 10, 20, 40, or 80 μg of CpG 1, with the remainder being the PBS solution.

The six- to eight-week-old female BALB/c mice were randomly grouped, each group contained 20 mice (10 mice in the PBS group), and the mice were immunized according to Table 6. On DO, each group of mice was immunized once in the inner thighs of the hind limbs separately, with an injection volume of 0.1 mL. The group immunized with the PBS solution was used as the control group. Blood was sampled from mice at the specified time points, 200-300 μL of blood was sampled from each mouse each time, serum was isolated, the geometric mean titers of 4 antibodies (anti-A1 antigen antibody, anti-A3 antigen antibody, anti-Bv antigen antibody, and anti-By antigen antibody) in the serum of mice were detected by ELISA, and the antibody titer was expressed as the geometric mean titer (GMT). Grouping of the mice is shown in Table 6.

TABLE 6
Grouping of Mice

| | | Antigen | CpG | Number |
|---|---|---|---|---|
| Groups and Names | | (μg/valence/dose) | (μg/dose) | of mice |
| 1 | PBS | — | — | 10 |
| 2 | QIV | 3.5 | — | 20 |
| 3 | QIV + CpG 1 | 3.5 | 10 | 20 |
| 4 | QIV + CpG 1 | 3.5 | 20 | 20 |
| 5 | QIV + CpG 1 | 3.5 | 40 | 20 |
| 6 | QIV + CpG 1 | 3.5 | 80 | 20 |

### 3. Results:

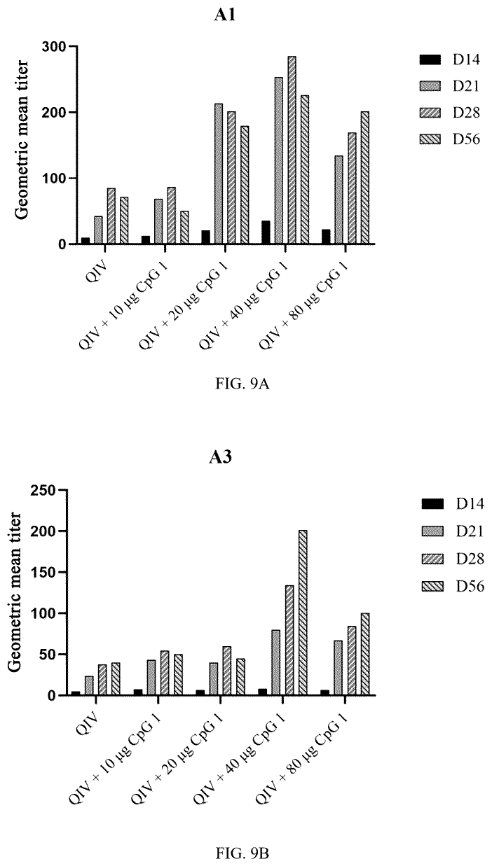

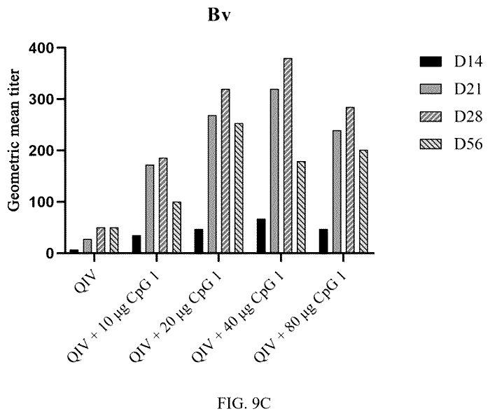

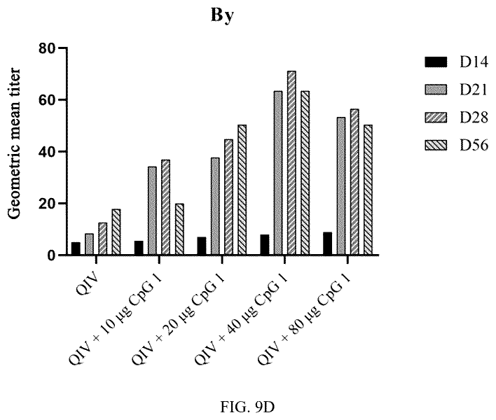

Referring to FIG. 9A to FIG. 9D, it can be learned from the figures that the CpG 1 can quickly induce the production of protective antibodies; significantly increase the antibody titer of the quadrivalent influenza vaccine (QIV), and maintain a high antibody titer level for a long period; and the immunity enhancement effect of the CpG 1 is dose-dependent within a certain range, showing a trend of initially increasing and then decreasing with increasing dose of CpG 1. When the dosage of the CpG 1 is 40 μg per dose per mouse, the immunostimulatory effect of the CpG 1 is the strongest.

Only several examples of the present invention are described in the above embodiments. The descriptions are relatively detailed and specific, but it cannot be construed as a limitation on the patent scope of the present invention. It should be noted that persons of ordinary skills in the art can make several modifications and improvements without departing from a concept of the present invention. Such modifications and improvements all belong to the protection scope of the present invention. Therefore, the scope of protection conferred by the patent shall be determined by the content of the accompanying claims, the description and the drawings serving for interpretation purposes.

1. A CpG oligonucleotide having a sequence as set forth in SEQ ID NO: 1.

2. The CpG oligonucleotide of claim 1, comprising a chemical modification.

3. The CpG oligonucleotide of claim 2, wherein the chemical modification is a modification of one or more phosphate ester groups.

4. The CpG oligonucleotide of claim 3, wherein the modification of the one or more phosphate ester groups comprises one or more of an internucleotide phosphorothioate ester linkage, a methylphosphonate linkage, and a boranophosphate linkage.

5. An immunostimulatory composition comprising the CpG oligonucleotide of claim 1.

6. The immunostimulatory composition of claim 5, further comprising an adjuvant other than the CpG oligonucleotide.

7. The immunostimulatory composition of claim 6, wherein the adjuvant comprises one or more of alum, complete Freund's adjuvant, incomplete Freund's adjuvant, squalene, squalane, muramyl dipeptide, MF59, AS03, AS04, 3-O-desacyl-4′-monophosphoryl lipid A, flagellin, Poly(I:C), and aluminum and calcium salts.

8. The immunostimulatory composition of claim 5, further comprising at least one antigen.

9. The immunostimulatory composition of claim 8, wherein the antigen is a tumor antigen, a viral antigen, a bacterial antigen, a fungal antigen, or a parasitic antigen.

10. The immunostimulatory composition of claim 9, wherein the immunostimulatory composition is a tumor therapeutic agent.

11. The immunostimulatory composition of claim 10, further comprising at least one antibody targeting the tumor antigen.

12. The immunostimulatory composition of claim 11, wherein the tumor antigen is selected from the group consisting of the following antigens or functional fragments thereof:
α-fetoprotein, α-actinin-4, A3, antigen specific to A33 antibodies, ART-4, B7, Ba 733, BAGE, BrE3 antigen, BMCA, CA125, CAMEL, CAP-1, carbonic anhydrase IX, CASP-8/m, CCL19, CCL21, CD1, CD1a, CD2, CD3, CD4, CD5, CD8, CD11A, CD14, CD15, CD16, CD18, CD19, CD21, CD23, CD25, CD29, CD30, CD32b, CD37, CD44, CD45, CD46, CD52, CD54, CD55, CD59, CD64, CD66a-e, CD67, CD70, CD70L, CD74, CD79a, CD79b, CD80, CD83, CD95, CD126, CD132, CD133, CD138, CD147, CD154, CDC27, CDK-4/m, CDKN2A, CLDN family proteins, CXCR4, CXCR7, CXCL12, HIF-1a, colon-specific antigen p, CEA, CEACAM-6, c-Met, DAM, EGFR, EGFRVIII, EGP-1, EGP-2, ELF2-M, Ep-CAM, fibroblast activation protein α, fibroblast growth factor, Flt-1, Flt-3, folate receptor, G250 antigen, GAGE, GD2, gp100, GRO-β, HLA-DR, HM1.24, human chorionic gonadotropin and a subunit thereof, HMGB-1, hypoxia-inducible factor, HSP70-2M, HST-2, Ia, IGF-1R, IFN-γ, IFN-α, IFN-β, IFN-λ, IL-4R, IL-6R, IL-13R, IL-15R, IL-17R, IL-18R, IL-2, IL-6, IL-8, IL-12, IL-15, IL-17, IL-18, IL-23, IL-25, insulin-like growth factor 1, KC4 antigen, KS-1 antigen, KS1-4, Le-Y, LDR/FUT, macrophage migration inhibitory factor, MAGE, MAGE-3, MART1, MART-2, NY-ESO-1, TRAG-3, mCRP, MCP-1, MIP-1α, MIP-1β, MIF, MUC1, MUC2, MUC3, MUC4, MUC5ac, MUC13, MUC16, MUM-1/2, MUM-3, NCA66, NCA95, NCA90, mesothelin, mucin in pancreatic cancer, prostate stem cell antigen, placenta growth factor, p53, PLAGL2, prostatic acid phosphatase, trophoblast cell surface antigen 2, PSA, PRAME, PSMA, PIGF, ILGF, ILGF-1R, IL-6, IL-25, RS5, RANTES, T101, SAGE, S100, survivin, survivin-2B, TAC, TAG-72, tenascin, TRAIL receptor, TNF-α, Tn antigen, Thomsen-Friedenreich antigen, tumor necrosis antigen, VEGFR, ED-B fibronectin, WT-1, 17-1A antigen, complement factor C3, C3a, C3b, C5a, C5, angiogenesis markers, bc1-2, bc1-6, and Kras.

13. The immunostimulatory composition of claim 5, further comprising one or more of an immune cell therapeutic drug, a chemical drug, a substance for promoting mucosal immune absorption or mucosal adhesion, an immunomodulator, a ligand for a pattern recognition receptor, and a pharmaceutically acceptable salt or excipient.

14. The immunostimulatory composition of claim 5, wherein the immunostimulatory composition is a vaccine adjuvant.

15. The immunostimulatory composition of claim 14, wherein the CpG oligonucleotide has a concentration of from 1-2000 μg/mL.

16. The immunostimulatory composition of claim 15, further comprising an aluminum salt adjuvant.

17. The immunostimulatory composition of claim 16, wherein the aluminum salt adjuvant has a concentration of from 100 μg/mL to 1 mg/mL.

18. The immunostimulatory composition of claim 8, wherein the immunostimulatory composition is a vaccine.

19. The immunostimulatory composition of claim 18, wherein the at least one antigen is a tumor antigen, a viral antigen, a bacterial antigen, a fungal antigen, or a parasitic antigen.

20. The immunostimulatory composition of claim 19, wherein the viral antigen is a coronavirus antigen, a herpes virus antigen, a hepatitis virus antigen, or an influenza virus antigen.

21. The immunostimulatory composition of claim 20, wherein the viral antigen is a novel coronavirus antigen, a varicella-zoster virus antigen, a hepatitis B virus antigen, an influenza A virus antigen, or an influenza B virus antigen.

22. The immunostimulatory composition of claim 19, wherein the mass ratio of the viral antigen to the CpG oligonucleotide is from 10:1 to 1:150.

23. The immunostimulatory composition of claim 19, wherein the CpG oligonucleotide has a concentration of from 1-2000 μg/mL and the viral antigen has a concentration of from 1-100 μg/mL.

24. A delivery system, comprising:
(i) the CpG oligonucleotide of claim 1; and
(ii) a delivery medium.

25. The delivery system of claim 24, wherein the delivery medium comprises one or more liposomes, one or more exosomes, one or more microvesicles, one or more dendrimers, one or more nanocomposites, one or more nanogels, one or more gold nanoparticles, polylactic-co-glycolic acid, one or more cell-penetrating peptides, and a combination thereof.

26. A method for regulating immune cell activity, the method comprising using a medicant comprising the CpG oligonucleotide of claim 1 in vivo or in vitro.

27. The method of claim 26, wherein the immune cells are one or more of macrophages, lymphocytes, and dendritic cells.

28. The method of claim 26, wherein regulating the immune cell activity is stimulating the immune cells to release inflammatory factors.

29. The method of claim 28, wherein the inflammatory factor comprises at least one of IFN-α, TNF-α, and IL-6.

30. A method comprising using a medicant comprising the CpG oligonucleotide of claim 1 for treating and/or preventing a disease in a subject, the disease being selected from the group consisting of a tumor, a viral infection, a bacterial infection, a fungal infection, a parasitic infection, a chemotherapy side effect, fatigue, immunosuppression, and a low immune response to an antigen.

31-38. (canceled)

39. A method for inducing a TLR9-mediated immune response in a subject, the method comprising administering an effective amount of the CpG oligonucleotide of claim 1 to the subject.

40. The method of claim 39, wherein the effective amount of the CpG oligonucleotide is administered via an intravenous route, an intramuscular route, an intramammary route, an intradermal route, an intraperitoneal route, a subcutaneous route, spraying, an intra-ovular route, a mucous route, a transdermal route, immersion, an oral route, an intraocular route, an intratracheal route, or an intranasal route.

41. (canceled)

42. The method of claim 39, wherein the disease related to TLR9-mediated immune response is at least one of a tumor, a viral infection, a bacterial infection, a fungal infection, a parasitic infection, a chemotherapy side effect, fatigue, immunosuppression, or a low immune response of the subject to an antigen.

43. The method of claim 39, wherein the CpG oligonucleotide of claim 1 is used as a vaccine adjuvant or a vaccine.

44. The method of claim 43, wherein the vaccine adjuvant or the vaccine is for a coronavirus, a herpes virus, a hepatitis virus, or an influenza virus.

45. The method of claim 44, wherein the vaccine adjuvant or the vaccine is for novel coronavirus, a varicella-zoster virus, a hepatitis B virus, an influenza A virus, or an influenza B virus.

46. The method of claim 39, wherein the subject is a mammal.

47. The method of claim 46, wherein the subject is a primate.

48. The method of claim 47, wherein the subject is a human.

49. The immunostimulatory composition of claim 15, wherein the CpG oligonucleotide has a concentration of from comprising 5-500 μg/mL.

50. The immunostimulatory composition of claim 16, wherein the aluminum salt adjuvant is an aluminum hydroxide adjuvant.

51. The immunostimulatory composition of claim 17, wherein the aluminum salt adjuvant has a concentration of from 400 μg/mL to 800 μg/mL.

52. The immunostimulatory composition of claim 21, wherein the viral antigen is a type A1 influenza virus antigen, type A3 influenza virus antigen, type Bv influenza virus antigen, or type By influenza virus antigen.

53. The immunostimulatory composition of claim 22, wherein the mass ratio of the viral antigen to the CpG oligonucleotide is from 5:1 to 1:50.

54. A delivery system, comprising:
(i) the immunostimulatory composition of claim 5; and
(ii) a delivery medium.

55. The method of claim 45, wherein the vaccine adjuvant or the vaccine is for type A1 influenza virus, type A3 influenza virus, type Bv influenza virus, or type By influenza virus.
